SEQUENCE LISTING (81 sequences)

SEQ ID NO: 1 - DNA, 20 bp, synthetic construct
  source 1..20: mol_type = other DNA
gatccaccagcacgagcagg

SEQ ID NO: 2 - DNA, 20 bp, synthetic construct
  source 1..20: mol_type = other DNA
ggatccaccagcacgagcag

SEQ ID NO: 3 - DNA, 20 bp, synthetic construct
  source 1..20: mol_type = other DNA
gtgatccaccagcacgagca

SEQ ID NO: 4 - DNA, 20 bp, synthetic construct
  source 1..20: mol_type = other DNA
gctgatccaccagcacgagc

SEQ ID NO: 5 - DNA, 20 bp, synthetic construct
  source 1..20: mol_type = other DNA
ggctgatccaccagcacgag

SEQ ID NO: 6 - DNA, 20 bp, synthetic construct
  source 1..20: mol_type = other DNA
tgatccaccagcacgagcag

SEQ ID NO: 7 - DNA, 20 bp, synthetic construct
  source 1..20: mol_type = other DNA
ctgatccaccagcacgagca

SEQ ID NO: 8 - DNA, 20 bp, synthetic construct
  source 1..20: mol_type = other DNA
tgctgatccaccagcacgag

SEQ ID NO: 9 - DNA, 20 bp, synthetic construct
  source 1..20: mol_type = other DNA
gctaggtgggaacctgctcg

SEQ ID NO: 10 - DNA, 20 bp, synthetic construct
  source 1..20: mol_type = other DNA
gtctaggtgggaacctgctc

SEQ ID NO: 11 - DNA, 20 bp, synthetic construct
  source 1..20: mol_type = other DNA
gatctaggtgggaacctgct

SEQ ID NO: 12 - DNA, 20 bp, synthetic construct
  source 1..20: mol_type = other DNA
gcatctaggtgggaacctgc

SEQ ID NO: 13 - DNA, 20 bp, synthetic construct
  source 1..20: mol_type = other DNA
gtcatctaggtgggaacctg

SEQ ID NO: 14 - DNA, 20 bp, synthetic construct
  source 1..20: mol_type = other DNA
gctcatctaggtgggaacct

SEQ ID NO: 15 - DNA, 20 bp, synthetic construct
  source 1..20: mol_type = other DNA
ggctcatctaggtgggaacc

SEQ ID NO: 16 - DNA, 20 bp, synthetic construct
  source 1..20: mol_type = other DNA
gacctagatgagcaaggatt

SEQ ID NO: 17 - DNA, 20 bp, synthetic construct
  source 1..20: mol_type = other DNA
gcacctagatgagcaaggat

SEQ ID NO: 18 - DNA, 20 bp, synthetic construct
  source 1..20: mol_type = other DNA
gccacctagatgagcaagga

SEQ ID NO: 19 - DNA, 20 bp, synthetic construct
  source 1..20: mol_type = other DNA
gcccacctagatgagcaagg

SEQ ID NO: 20 - DNA, 20 bp, synthetic construct
  source 1..20: mol_type = other DNA
gtcccacctagatgagcaag

SEQ ID NO: 21 - DNA, 20 bp, synthetic construct
  source 1..20: mol_type = other DNA
gttcccacctagatgagcaa

SEQ ID NO: 22 - DNA, 20 bp, synthetic construct
  source 1..20: mol_type = other DNA
ggttcccacctagatgagca

SEQ ID NO: 23 - DNA, 20 bp, synthetic construct
  source 1..20: mol_type = other DNA
tctaggtgggaacctgctcg

SEQ ID NO: 24 - DNA, 20 bp, synthetic construct
  source 1..20: mol_type = other DNA
atctaggtgggaacctgctc

SEQ ID NO: 25 - DNA, 20 bp, synthetic construct
  source 1..20: mol_type = other DNA
catctaggtgggaacctgct

SEQ ID NO: 26 - DNA, 20 bp, synthetic construct
  source 1..20: mol_type = other DNA
tcatctaggtgggaacctgc

SEQ ID NO: 27 - DNA, 20 bp, synthetic construct
  source 1..20: mol_type = other DNA
ctcatctaggtgggaacctg

SEQ ID NO: 28 - DNA, 20 bp, synthetic construct
  source 1..20: mol_type = other DNA
tgctcatctaggtgggaacc

SEQ ID NO: 29 - DNA, 20 bp, synthetic construct
  source 1..20: mol_type = other DNA
cacctagatgagcaaggatt

SEQ ID NO: 30 - DNA, 20 bp, synthetic construct
  source 1..20: mol_type = other DNA
ccacctagatgagcaaggat

SEQ ID NO: 31 - DNA, 20 bp, synthetic construct
  source 1..20: mol_type = other DNA
cccacctagatgagcaagga

SEQ ID NO: 32 - DNA, 20 bp, synthetic construct
  source 1..20: mol_type = other DNA
tcccacctagatgagcaagg

SEQ ID NO: 33 - DNA, 20 bp, synthetic construct
  source 1..20: mol_type = other DNA
ttcccacctagatgagcaag

SEQ ID NO: 34 - DNA, 20 bp, synthetic construct
  source 1..20: mol_type = other DNA
gtaccaatgataagctgctc

SEQ ID NO: 35 - DNA, 20 bp, synthetic construct
  source 1..20: mol_type = other DNA
gttaccaatgataagctgct

SEQ ID NO: 36 - DNA, 20 bp, synthetic construct
  source 1..20: mol_type = other DNA
gcttaccaatgataagctgc

SEQ ID NO: 37 - DNA, 20 bp, synthetic construct
  source 1..20: mol_type = other DNA
gccttaccaatgataagctg

SEQ ID NO: 38 - DNA, 20 bp, synthetic construct
  source 1..20: mol_type = other DNA
gtccttaccaatgataagct

SEQ ID NO: 39 - DNA, 20 bp, synthetic construct
  source 1..20: mol_type = other DNA
gatccttaccaatgataagc

SEQ ID NO: 40 - DNA, 20 bp, synthetic construct
  source 1..20: mol_type = other DNA
ggatccttaccaatgataag

SEQ ID NO: 41 - DNA, 20 bp, synthetic construct
  source 1..20: mol_type = other DNA
ttaccaatgataagctgctc

SEQ ID NO: 42 - DNA, 20 bp, synthetic construct
  source 1..20: mol_type = other DNA
cttaccaatgataagctgct

SEQ ID NO: 43 - DNA, 20 bp, synthetic construct
  source 1..20: mol_type = other DNA
ccttaccaatgataagctgc

SEQ ID NO: 44 - DNA, 20 bp, synthetic construct
  source 1..20: mol_type = other DNA
tccttaccaatgataagctg

SEQ ID NO: 45 - DNA, 20 bp, synthetic construct
  source 1..20: mol_type = other DNA
atccttaccaatgataagct

SEQ ID NO: 46 - DNA, 20 bp, synthetic construct
  source 1..20: mol_type = other DNA
agatccttaccaatgataag

SEQ ID NO: 47 - DNA, 22 bp, synthetic construct
  source 1..22: mol_type = other DNA
acacacatgatggttgtcgtct

SEQ ID NO: 48 - DNA, 23 bp, synthetic construct
  source 1..23: mol_type = other DNA
gctttctcacaacagttgcagtt

SEQ ID NO: 49 - DNA, 27 bp, synthetic construct
  source 1..27: mol_type = other DNA
ccagcaagtagataacaagtttattgc

SEQ ID NO: 50 - DNA, 25 bp, synthetic construct
  source 1..25: mol_type = other DNA
ctctagcggaggaattagagtagaa

SEQ ID NO: 51 - DNA, 10 bp, synthetic construct
  source 1..10: mol_type = other DNA
gctcatctag

SEQ ID NO: 52 - DNA, 10 bp, synthetic construct
  source 1..10: mol_type = other DNA
gctcatctaa

SEQ ID NO: 53 - DNA, 10 bp, synthetic construct
  source 1..10: mol_type = other DNA
gctcatctcg

SEQ ID NO: 54 - protein, 12 aa, synthetic construct
  VARIANT 5..6: note = X can be any amino acid
  VARIANT 8..11: note = X can be any amino acid
DQHAXXEXXXXE

SEQ ID NO: 55 - protein, 12 aa, synthetic construct
DQHAAHERIRLE

SEQ ID NO: 56 - DNA, 20 bp, synthetic construct
  source 1..20: mol_type = other DNA
acgagtagatccacccttgg

SEQ ID NO: 57 - DNA, 20 bp, synthetic construct
  source 1..20: mol_type = other DNA
aacgagtagatccacccttg

SEQ ID NO: 58 - DNA, 20 bp, synthetic construct
  source 1..20: mol_type = other DNA
gaacgagtagatccaccctt

SEQ ID NO: 59 - DNA, 20 bp, synthetic construct
  source 1..20: mol_type = other DNA
ggaacgagtagatccaccct

SEQ ID NO: 60 - DNA, 20 bp, synthetic construct
  source 1..20: mol_type = other DNA
aggaacgagtagatccaccc

SEQ ID NO: 61 - DNA, 20 bp, synthetic construct
  source 1..20: mol_type = other DNA
taggaacgagtagatccacc

SEQ ID NO: 62 - DNA, 20 bp, synthetic construct
  source 1..20: mol_type = other DNA
ttaggaacgagtagatccac

SEQ ID NO: 63 - DNA, 20 bp, synthetic construct
  source 1..20: mol_type = other DNA
gagcacgaccacctagtcgt

SEQ ID NO: 64 - DNA, 20 bp, synthetic construct
  source 1..20: mol_type = other DNA
cgagcacgaccacctagtcg

SEQ ID NO: 65 - DNA, 20 bp, synthetic construct
  source 1..20: mol_type = other DNA
acgagcacgaccacctagtc

SEQ ID NO: 66 - DNA, 20 bp, synthetic construct
  source 1..20: mol_type = other DNA
gacgagcacgaccacctagt

SEQ ID NO: 67 - DNA, 20 bp, synthetic construct
  source 1..20: mol_type = other DNA
ggacgagcacgaccacctag

SEQ ID NO: 68 - DNA, 20 bp, synthetic construct
  source 1..20: mol_type = other DNA
gaatagtaaccattcctaga

SEQ ID NO: 69 - DNA, 20 bp, synthetic construct
  source 1..20: mol_type = other DNA
cgaatagtaaccattcctag

SEQ ID NO: 70 - DNA, 20 bp, synthetic construct
  source 1..20: mol_type = other DNA
tcgaatagtaaccattccta

SEQ ID NO: 71 - DNA, 20 bp, synthetic construct
  source 1..20: mol_type = other DNA
gtcgaatagtaaccattcct

SEQ ID NO: 72 - DNA, 20 bp, synthetic construct
  source 1..20: mol_type = other DNA
cgtcgaatagtaaccattcc

SEQ ID NO: 73 - DNA, 20 bp, synthetic construct
  source 1..20: mol_type = other DNA
tcgtcgaatagtaaccattc

SEQ ID NO: 74 - DNA, 20 bp, synthetic construct
  source 1..20: mol_type = other DNA
ctcgtcgaatagtaaccatt

SEQ ID NO: 75 - protein, 23 aa, synthetic construct
GNLLVLVDQHAAHERIRLEQLII

SEQ ID NO: 76 - DNA, 107 bp, synthetic construct
  source 1..107: mol_type = other DNA
cacaatccttgctcatctaggtgggaacctgctcgtgctggtggatcagcacgctgcccatgagcgtatacgtctggagcagcttatcattggtaaggatctgtttg

SEQ ID NO: 77 - DNA, 107 bp, synthetic construct
  source 1..107: mol_type = other DNA
gtgttaggaacgagtagatccacccttggacgagcacgaccacctagtcgtgcgacgggtactcgcatatgcagacctcgtcgaatagtaaccattcctagacaaac

SEQ ID NO: 78 - DNA, 107 bp, synthetic construct
  source 1..107: mol_type = other DNA
cacaatccttgctcatctaagtgggaacctgctcgtgctggtgaatcagcacgctgcccatgagcgtatacgtctggagcagcttatcattgataaggatctgtttg

SEQ ID NO: 79 - DNA, 107 bp, synthetic construct
  source 1..107: mol_type = other DNA
gtgttaggaacgagtagattcacccttggacgagcacgaccacttagtcgtgcgacgggtactcgcatatgcagacctcgtcgaatagtaactattcctagacaaac

SEQ ID NO: 80 - DNA, 107 bp, synthetic construct
  source 1..107: mol_type = other DNA
cacaatccttgctcatctgggtgggaacctgctcgtgctggtgaatcagcacgctgcccatgagcgtatacgtctggagcagcttatcattggcaaggatctgtttg

SEQ ID NO: 81 - DNA, 107 bp, synthetic construct
  source 1..107: mol_type = other DNA
gtgttaggaacgagtagacccacccttggacgagcacgaccacttagtcgtgcgacgggtactcgcatatgcagacctcgtcgaatagtaaccgttcctagacaaac

(19) United States
(12) Patent Application Publication
(10) Pub. No.: US 2026/0240960 A1
(43) Pub. Date: Aug. 20, 2026

(54) COMPOSITIONS AND METHODS FOR TREATING TRINUCLEOTIDE REPEAT DISORDERS

(71) Applicant: The General Hospital Corporation, Boston, MA (US)

(72) Inventors: Ricardo Mouro Pinto, Boston, MA (US); Benjamin Kleinstiver, Boston, MA (US)

(21) Appl. No.: 19/106,116

(22) PCT Filed: Aug. 24, 2023
(86) PCT No.: PCT/US2023/072851

§ 371 Date: Feb. 24, 2025

Related U.S. Application Data
(60) Provisional application No. 63/400,443, filed on Aug. 24, 2022.

(51) Int. Cl. A61K 38/46; A61K 31/7088; C12N 9/22; C12N 15/11; C12N 15/90

(52) U.S. Cl. CPC A61K 38/465; A61K 31/7088; C12N 9/226; C12N 15/11; C12N 15/907; C12N 2310/20

(57) ABSTRACT

Methods and compositions for reducing expansion of nucleotide repeats in a cell, comprising base editors and a guide RNA (gRNA) that directs the Cas-based enzyme to the splice acceptor site for mutL homolog 3 (MLH3) exon 7 or to the MLH3 endonuclease domain.

### CLAIM OF PRIORITY

This application claims the benefit of U.S. Provisional Application Ser. No. 63/400,443, filed on Aug. 24, 2022. The entire contents of the foregoing are incorporated herein by reference.

### FEDERALLY SPONSORED RESEARCH OR DEVELOPMENT

This invention was made with Government support under Grant No. NS126420 awarded by the National Institutes of Health. The Government has certain rights in the invention.

### TECHNICAL FIELD

Methods and compositions for reducing expansion of nucleotide repeats in a cell, comprising base editors and a guide RNA (gRNA) that directs the Cas-based enzyme to the splice acceptor site for mutL homolog 3 (MLH3) exon 7, or MLH3 or the endonuclease domain.

### BACKGROUND

Various diseases, including but not limited to neurological and motor diseases, can result from expanded nucleotide repeat sequences within human genes. These expanded sequences can lead to a variety of consequences including decreased gene expression, the production of dominant negative proteins, RNA transcripts or protein aggregates, and other molecular pathologies. For example, Huntington's disease (HD) is a fatal neurodegenerative disorder caused by a mutant Huntingtin gene (HTT) harboring an expanded CAG repeat. The large CAG tract continues to expand in somatic tissues and contributes to HD pathogenesis, with larger rates of expansion attributed to earlier age of onset and faster disease progression.

### SUMMARY

Provided herein are methods for reducing expansion of nucleotide repeats, e.g., an expansion of CAG trinucleotide repeats in a huntingtin (HTT) gene, in a cell. The methods include contacting the cell with or expressing in the cell a base editor enzyme and a guide RNA (gRNA) that directs the base editor to a splice acceptor site (e.g., to introduce a single base mutation at the MLH3 exon 7 splice acceptor and/or splice donor site to promote skipping of exon 7) or endonuclease domain (e.g., to convert the Aspartic Acid (D) at amino acid 1223 to an Asparagine (N)) for mutL homolog 3 (MLH3) exon 7 in an amount sufficient to reduce the expansion of nucleotide repeats in the cell.

Also provided herein are methods for treating a subject who has a condition associated with nucleotide repeat expansion, e.g., CAG nucleotide repeat expansion in a HTT gene. The methods include administering to the subject a therapeutically effective amount of a base editor and a guide RNA that directs the base editor to MLH3, preferably wherein the gRNA binds to an exon/repeat border (e.g., to introduce a single base mutation at the MLH3 exon 7 splice acceptor and/or splice donor site to promote skipping of exon 7) or endonuclease domain (e.g., to convert the Aspartic Acid (D) at amino acid 1223 to an Asparagine (N)), in an amount sufficient to reduce the expansion of nucleotide repeats in the cell. In some embodiments, the base editor and gRNA are administered to the CNS, e.g., brain or spinal cord of the subject (e.g., via ICV, cisternae magna, or intrathecal administration), or administered systemically to the subject.

In some embodiments, the base editor comprises a Cas9 DNA binding domain, optionally wild type SpCas9, SpG, SpRY, SpNG, SpNRRH, VRQR, SpNRCH, or SpNRTH.

In some embodiments, the base editor comprises a nicking or catalytically inactive wild type SpCas9 DNA binding domain, SpG, SpRY, SpNG, SpNRRH, VRQR, SpNRCH, or SpNRTH and a UGI or TadA.

In some embodiments, the gRNA comprises a spacer sequence listed in Table 1, optionally truncated to 19, 18, or 17 nt by removing 1, 2, or 3 bases from the 3′ PAM distal end of the spacer. In some embodiments, the gRNA comprises a spacer sequence SA-A4, SA-A5, or SA-A6; or any of DN-C1 to DN-C5; optionally truncated to 19, 18, or 17 nt by removing 1, 2, or 3 bases from the 3′ PAM distal end of the spacer.

In some embodiments, the base editor and gRNA are: ABE8e-SpG and SA-A6; ABE8e-SpG and SA-A4; ABE8e-SpRY and SA-A5; ABE8e-SpNRCH and SA-A4; ABE8e-SpNRCH and SA-A6; ABE8e-SpNG and SA-A5; ABE8e-SpNG and SA-A6; ABE8e-SpNRTH and SA-A4; or ABE8e-SpNRTH and SA-A6; or any of DN-C1 to DN-C5 with SpRY.

In some embodiments, the base editor and gRNA are administered in an expression vector, e.g., a plasmid or viral vector; are administered as mRNA; or are administered as RNPs.

Additionally, provided herein are compositions comprising a base editor and gRNA, wherein the gRNA comprises a spacer sequence having 17-20 nts of SA-A4, SA-A5, or SA-A6, or any of DN-C1 to DN-C5, optionally wherein the base editor comprises Cas9, optionally wild type SpCas9, SpG, SpRY, VRQR, SpNG, SpNRRH, SpNRCH, or SpNRTH or nicking or catalytically inactive wild type SpCas9, SpG, SpRY, VRQR, SpNG, SpNRRH, SpNRCH, or SpNRTH and a UGI or TadA. In some embodiments, the base editor and gRNA are: ABE8e-SpG and SA-A6; ABE8e-SpG and SA-A4; ABE8e-SpRY and SA-A5; ABE8e-SpNRCH and SA-A4; ABE8e-SpNRCH and SA-A6; ABE8e-SpNG and SA-A5; ABE8e-SpNG and SA-A6; ABE8e-SpNRTH and SA-A4; or ABE8e-SpNRTH and SA-A6; or any of DN-C1 to DN-C5 with SpRY. In some embodiments, the base editor and gRNA are in ribonucleoprotein complexes.

Also provided herein are nucleic acids encoding a base editor and gRNA, preferably wherein the gRNA comprises a spacer sequence having 17-20 nts of SA-A4, SA-A5, or SA-A6, or any of DN-C1 to DN-C5, optionally wherein the base editor comprises Cas9, optionally wild type SpCas9, SpG, SpRY, VRQR, SpNG, SpNRRH, SpNRCH, or SpNRTH or nicking or catalytically inactive wild type SpCas9, SpG, SpRY, VRQR, SpNG, SpNRRH, SpNRCH, or SpNRTH and a UGI or TadA. In some embodiments, the base editor and gRNA are: ABE8e-SpG and SA-A6; ABE8e-SpG and SA-A4; ABE8e-SpRY and SA-A5; ABE8e-SpNRCH and SA-A4; ABE8e-SpNRCH and SA-A6; ABE8e-SpNG and SA-A5; ABE8e-SpNG and SA-A6; ABE8e-SpNRTH and SA-A4; or ABE8e-SpNRTH and SA-A6; or any of DN-C1 to DN-C5 with SpRY.

In some embodiments, the nucleic acid is in an expression vector. In some embodiments, the expression vector is a plasmid or viral vector as described herein, and optionally further comprises at least one promoter that drives expression of the base editor and gRNA.

In some embodiments, the nucleic acid comprises mRNA encoding the base editor and gRNA.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF DRAWINGS

FIGS. 1A-C are schematics illustrating the mutL homolog 3 (MLH3) exon 7 splicing and the different base editing strategies targeting the endonuclease domain. 1A, MLH3 exon 7 codes for the endonuclease domain. Through alternative splicing, MLH3 is naturally expressed as two variants: variant 1 includes exon 7 and generates an MLH3 protein with endonuclease domain which causes somatic trinucleotide repeat expansions; variant 2 does not contain exon 7 and generates an MLH3 protein without endonuclease domain, which prevents trinucleotide repeat expansions. 1, Using ABE and/or CBE base editors guided to the MLH3 exon 7 splice acceptor motif, the splice acceptor motif can be disrupted and cause splice exclusion of exon 7. This results in expression of MLH3 protein without the endonuclease domain, which prevents trinucleotide repeat expansions. 1C, Using ABE and/or CBE base editors guided to the MLH3 exon 7 endonuclease motif, nonsynonymous mutations can be introduced in the endonuclease domain resulting in a catalytically inactive MLH3 protein, which prevents trinucleotide repeat expansions.

FIG. 2 is a schematic illustrating sequence and location of exemplary sgRNAs for mutL homolog 3 (MLH3) exon 7 base editing.

FIGS. 3A-B show the results of testing of base editing sgRNAs, using ABE and CBE, for A>G mutation and G>A mutation in the MLH3 exon 7 splice acceptor site, respectively. The transfections were performed in HEK293T, and editing evaluated after 7 days using next generation sequencing and CRISPResso2 bioinformatic analysis. 3A, Heatmap graph showing quantification of editing (%) at the respective ABE/CBE splice acceptor site. 3B, RT-PCR showing that editing of single base (A>G) in splice acceptor leads to complete elimination of MLH3 variant 1, which contains the endonuclease domain, and steady expression of MLH3 variant 2, which does not contain the endonuclease domain (i.e. single base edit in MLH3 exon 7 splice acceptor motif causes complete splice exclusion of exon 7 and consequent exclusion of MLH3 endonuclease domain).

FIG. 4 shows the results of testing base editing sgRNAs, using additional ABEs, for A>G mutation in the MLH3 exon 7 splice acceptor site. The transfections were performed in HEK293T, and editing evaluated after 72 hrs using next generation sequencing and CRISPResso2 bioinformatic analysis. Heatmap graph showing quantification of editing (%) at the respective ABE splice acceptor target site.

FIG. 5 shows the results of testing base editing sgRNAs, using CBE, for G>A mutation in the MLH3 exon 7 endonuclease domain motif, resulting in a D>N amino acid change that inactivates MLH3 endonuclease activity. The transfections were performed in HEK293T, and editing evaluated after 7 days using next generation sequencing and CRISPResso2 bioinformatic analysis. Heatmap graph showing quantification of editing (%) at the respective CBE splice acceptor target site.

### DETAILED DESCRIPTION

Huntington's disease (HD) is a dominantly inherited neurodegenerative disorder caused by an expanded CAG trinucleotide repeat in the huntingtin gene (HTT), encoding an extended polyglutamine tract in the huntingtin protein (Huntington's Disease Collaborative Research Group, Cell, 1993). The expanded CAG mutation ultimately results in neuronal dysfunction and death (Vonsattel et al., Journal of Neuropathology & Experimental Neurology, 1985), via mechanisms that are as yet unclear (McColgan and Tabrizi, European Journal of Neurology, 2018). The mutant CAG repeat undergoes further expansion in somatic cells in a CAG length-dependent and tissue- or cell-type-specific manner (Mouro Pinto et al., Human Molecular Genetics, 2020; Kennedy et al., Human Molecular Genetics, 2003; Shelbourne et al., Human Molecular Genetics, 2007; Gonitel et al., Proceedings of the National Academy of Sciences of the United States of America, 2008; Swami et al., Human Molecular Genetics, 2009). Somatic expansion is recapitulated in HD mouse models that also show progressive repeat tract lengthening over time (Lee et al., PLoS ONE, 2011; Kovalenko et al., PLoS ONE, 2012; Larson et al., Neurobiology of Disease, 2015; Ament et al., Human Molecular Genetics, 2017), with the striatum and liver showing high levels of expansion, particularly the medium spiny neurons and hepatocytes, respectively (Lee et al., PLoS ONE, 2011; Kovalenko et al., PLoS ONE, 2012). Studies of postmortem HD brains revealed that longer somatic CAG expansions are associated with an earlier age of disease onset (Swami et al., Human Molecular Genetics, 2009), with more recent studies showing correlations between age of onset or disease progression and the degree of somatic expansion in HD patient blood (Ciosi et al., EBioMedicine, 2019). Significantly, recent genome-wide association studies (GWAS) of over 9,000 HD patients have demonstrated that the rate of disease onset is determined by the length of the pure CAG repeat, rather than the length of the glutamine tract in huntingtin (GeM-HD, Cell, 2019). This is supported by additional studies (Ciosi et al., EBioMedicine, 2019; Wright et al., American Journal of Human Genetics, 2019) and provides compelling support for a model in which the rate of somatic CAG expansion drives the rate of disease onset. Thus, therapies targeting somatic repeat expansions have the potential to delay disease onset and progression.

Genetic knockout studies in HD mouse models have shown that Msh2, Msh3, Mlh1, and Mlh3 genes in the DNA mismatch repair (MMR) pathway are required for somatic expansion (Wheeler et al., Human Molecular Genetics, 2003; Owen et al., Nature Structural and Molecular Biology, 2005; Dragileva et al., Neurobiology of Disease, 2009; Mouro Pinto et al., PLoS Genetics, 2013). Human GWAS have identified several genes in this pathway (MSH3, MLH1, PMS1, PMS2) to be associated with age at motor onset (GeM-HD, Cell, 2015; Cell, 2019), with MSH3 also showing association with a measure of HD progression (Moss et al., The Lancet Neurology, 2017). The mouse ortholog of an additional HD onset-modifier gene, FAN1 (GeM-HD, Cell, 2015; Cell, 2019), with no known function in the MMR pathway, suppressed somatic expansion in HD knock-in mice in a manner that was dependent on Mlh1 (Loupe et al., Human Molecular Genetics, 2020). Genetic variation in some human onset or progression modifier genes (MSH3, MLH1, MLH3, FAN1) also modified CAG expansion in HD patient blood or blood-derived cells (Ciosi et al., EBioMedicine, 2019; Flower et al., Brain, 2019; GeM-HD, Cell, 2019). Together, these data indicate that HD pathogenesis is modulated by naturally occurring variation in genes that act by altering the rate of somatic CAG expansion, and that genes driving this process are potential therapeutic targets for HD. MMR pathway genes also modify somatic expansion in mouse models and in mouse or human cell-based models of a number of other DNA repeat expansion disorders, including myotonic dystrophy type I (DM1) (van den Broek et al., Human Molecular Genetics, 2002; Savouret et al., EMBO Journal, 2003; Foiry et al., Human Genetics, 2006), Friedreich ataxia (FRDA) (Bourn et al., PLoS ONE, 2012; Halabi et al., Journal of Biological Chemistry, 2012; Ezzatizadeh et al., PLoS ONE, 2014; Halabi, Fuselier and Grabczyk, Nucleic Acids Research, 2018), and the fragile X-related disorders (FXD) (Lokanga, Zhao and Usdin, Human Mutation, 2014; Zhao et al., PLOS Genetics, 2016; PLOS Genetics, 2018; Hayward, Steinbach and Usdin, Nucleic acids research, 2020; Miller et al., PLOS Genetics, 2020). In addition, polymorphisms in two DNA repair genes (FAN1, PMS2) associated with HD onset were also associated with disease onset in the spinocerebellar ataxias (Bettencourt et al., Annals of Neurology, 2016). Thus, therapeutic strategies targeting MMR genes may be applicable to multiple repeat expansion diseases.

Previous genetic knockout studies in HttQ111 knock-in mice demonstrated that MLH3 is an essential component of the CAG repeat expansion process (Mouro Pinto et al., PLoS Genetics, 2013). MLH3 is known to play an important role in meiosis, but a relatively minor role in canonical MMR (Lipkin et al., Nature Genetics, 2002; Cannavo et al., Cancer Research, 2005; Charbonneau et al., Cancer Biology and Therapy, 2009). MLH3 contains an evolutionary conserved endonuclease domain (Kadyrov et al., Cell, 2006) that we postulated may be important in its CAG expansion-promoting role in HD (Mouro Pinto et al., PLoS Genetics, 2013), with inactivation or elimination of this domain hypothesized to eliminate or reduce somatic expansions.

The endonuclease domain of MLH3 is encompassed in the C-terminal domain of the protein, which interacts with MLH1 (Kondo, Horii and Fukushige, Nucleic Acids Research, 2001). A single D>N amino acid substitution in the first amino acid of the DQHA(X)2E(X4)E motif eliminates MLH3 endonuclease activity (Ranjha, Anand and Cejka, Journal of Biological Chemistry, 2014; Rogacheva et al., Journal of Biological Chemistry, 2014; Claeys Bouuaert and Keeney, PLOS Genetics, 2017; Kadyrova et al., Proceedings of the National Academy of Sciences of the United States of America, 2020). There is no evidence that this mutation alters the stability of MLH3 or its interaction with MLH1 (Nishant, Plys and Alani, Genetics, 2008; Toledo et al., PLoS Genetics, 2019; Hayward, Steinbach and Usdin, Nucleic acids research, 2020; Kadyrova et al., Proceedings of the National Academy of Sciences of the United States of America, 2020).

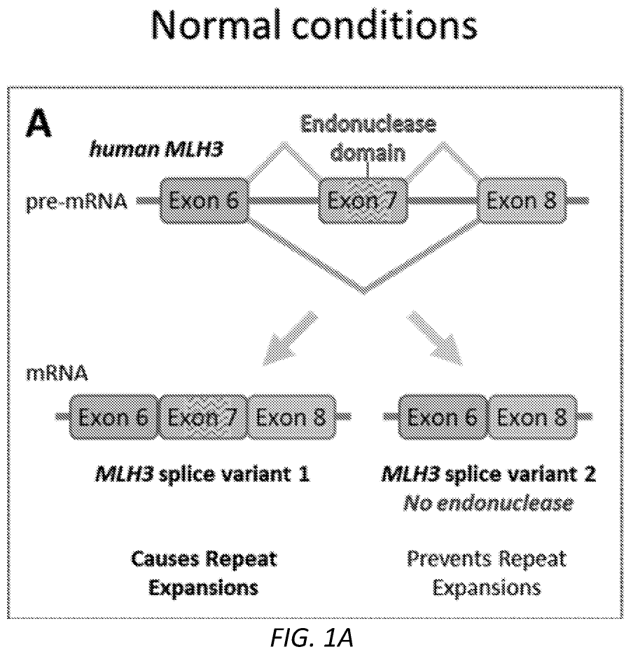

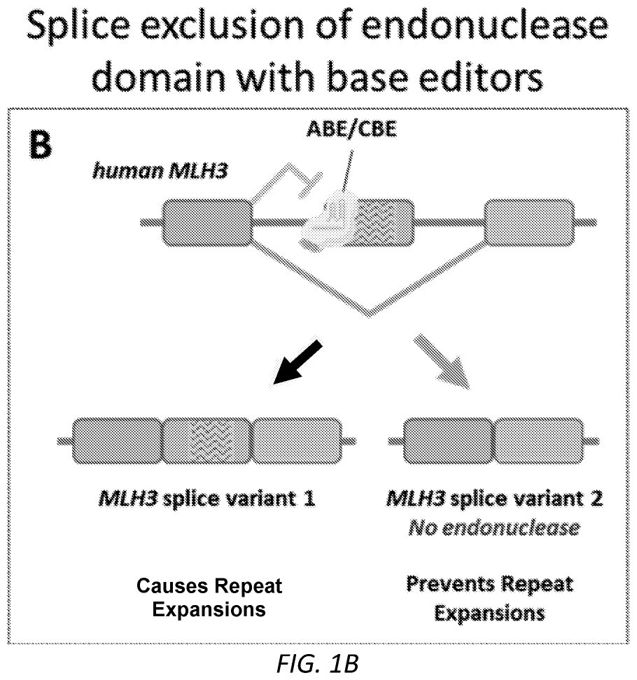

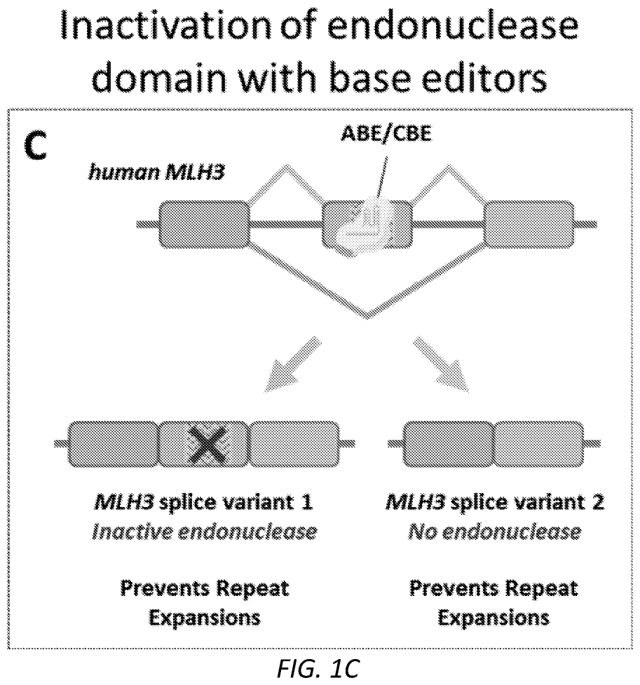

Interestingly, human MLH3 exists primarily as two protein isoforms: isoform 1 (UniProt Q9UHC1-1) is specified by splice variant 1 (CCDS32123) containing exon 7, which encodes the endonuclease domain, while isoform 2 (UniProt Q9UHC1-2) is specified by splice variant 2 (CCDS9837), lacking exon 7, and therefore has no endonuclease domain (FIG. 1A) (Roy et al., Nucleic Acids Research, 2021; Halabi, Fuselier and Grabczyk, Nucleic Acids Research, 2018; Yates et al., Nucleic Acids Research, 2020). MLH3 splice redirection from predominantly splice variant 1 to predominantly splice variant 2 can be achieved with splice switching oligonucleotides (SSOs) that mask the acceptor and donor sites that surround exon 7 of the MLH3 pre-mRNA (Roy et al., Nucleic Acids Research, 2021; Halabi, Fuselier and Grabczyk, Nucleic Acids Research, 2018).

Splice redirection of MLH3 to exclude the endonuclease domain resulted in reduced rates of GAA expansion in a human cell-based model and in Friedreich ataxia (FRDA) patient cells (Halabi, Fuselier and Grabczyk, Nucleic Acids Research, 2018). This indicated that the MLH3 endonuclease domain was important for GAA repeat expansion and that splice redirection could be used to reduce its contribution to the expansion process in FRDA. Additionally, point mutation in the MLH3 endonuclease domain (D1185N in mice, D1223N in humans) was found to abrogate expansion of the CGG repeat tract in a mouse embryonic cell-based model of Fragile X-related disorders (FXDs) (Hayward, Steinbach and Usdin, Nucleic acids research, 2020).

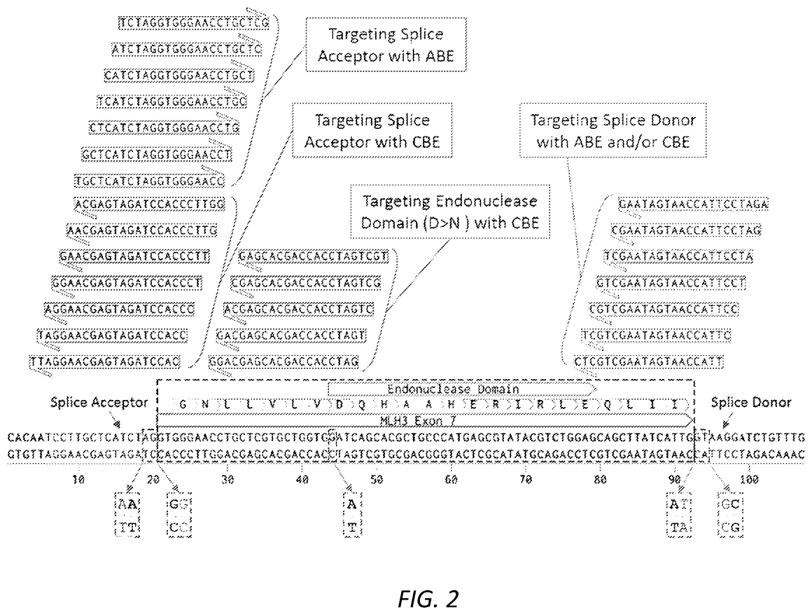

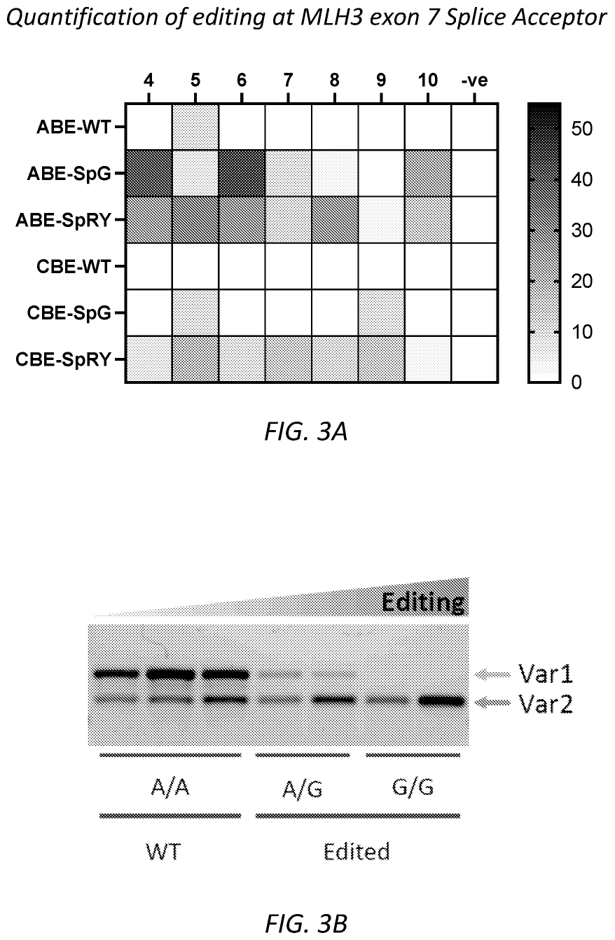

In HD, it was demonstrated that the MLH3 endonuclease domain is essential for somatic HTT CAG expansion in Huntington's disease, using both genetic studies and a pharmacological approach based on splice redirection (Roy et al., Nucleic Acids Research, 2021): 1) a single point mutation in the MLH3 endonuclease domain (D1185N in mice, D1223N in humans), predicted to abrogate endonuclease activity, eliminates somatic CAG expansion in the brain and peripheral tissues of HttQI11 HD mice, having an effect indistinguishable from a Mlh3 null mutation (Roy et al., 2021, FIG. 1); 2) systemic delivery of SSOs to redirect Mlh3 splicing to exclude the endonuclease domain suppresses somatic CAG expansion in peripheral tissues of HttQ111 HD mice (Roy et al., 2021, FIG. 2); and 3) MLH3 splice redirection with SSOs also effectively reduces CAG expansion in HD patient-derived primary fibroblast cells, in the absence of replication (Roy et al., 2021, FIG. 3). These data highlight the therapeutic potential of targeting the MLH3 endonuclease domain to slow HTT CAG repeat expansion, a strategy that may be applicable across multiple repeat expansion disorders.

Described herein are CRISPR base editing approaches to inactivate MLH3 endonuclease activity. These methods may work via introduction of nonsynonymous mutations (an alteration of the nucleotide sequence of a gene that changes the amino acid sequence of the protein) in the endonuclease domain (DQHAAHERIRLE) (FIG. 1C) or by permanent splice redirection to exclude the endonuclease domain (FIG. 1B). This is a promising therapeutic approach for trinucleotide repeat diseases by suppressing trinucleotide repeat expansion and potentially disease progression in Huntington disease and other MLH3 dependent expansions in repeat disorders.

The present methods can be used, e.g., to introduce nonsynonymous mutations at the MLH3 exon 7 endonuclease motif to inactivate its function. Specifically, the present methods can be used to introduce a single base mutation at the MLH3 exon 7 splice acceptor and/or splice donor site to promote skipping of exon 7, which encodes for the endonuclease domain, and therefore promote expression of MLH3 protein lacking the endonuclease domain. Alternatively, the present methods can be used to convert the Aspartic Acid (D) at amino acid 1223 to an Asparagine (N), which is known to ablate MLH3 endonuclease activity. The methods use adenosine base editors (ABEs) and/or cytosine base editors (CBEs) to recognize non-canonical PAM sites, e.g., base editors that include the SPG and/or SPRY Cas9 variants.

### Methods of Use

In some embodiments, the present methods and compositions can be used to suppress somatic expansions in Huntington's disease (HD)-associated CAG nucleotide expansions in a living cell or subject, or a cell or population of cells from a subject, who has HD associated with CAG nucleotide expansions, e.g., 36 or more CAG repeats in the HTT gene. Although exemplified on CAG repeats in HTT, the present methods can also be used in other repeat expansion diseases, e.g., as described in WO2022197857, particularly Fragile X-associated disorders (FXD) such as Fragile X syndrome (FXS), fragile X-associated tremor/ataxia syndrome (FXTAS), Friedreich ataxia (FRDA), Myotonic dystrophy type 1 and 2 (DM1, DM2), multiple Spinocerebellar ataxias (SCA1, 2, 3, 6, 7, 12, 17 and 27B), Amyotrophic Lateral Sclerosis (ALS), X-Linked Dystonia Parkinsonism (XDP), Fuchs' endothelial corneal dystrophy (FECD), spinal and bulbar muscular atrophy (SBMA) and dentatorubral-pallidoluysian atrophy (DRPLA). This is supported by prior demonstration that MLH3, and other mismatch repair genes, contribute to somatic repeat expansions of other disease-associated repeat loci, such as myotonic dystrophy type I (DM1) (van den Broek et al., Human Molecular Genetics, 2002; Savouret et al., EMBO Journal, 2003; Foiry et al., Human Genetics, 2006), Friedreich ataxia (FRDA) (Bourn et al., PLoS ONE, 2012; Halabi et al., Journal of Biological Chemistry, 2012; Ezzatizadeh et al., PLoS ONE, 2014; Halabi, Fuselier and Grabczyk, Nucleic Acids Research, 2018), the fragile X-related disorders (FXD) (Lokanga, Zhao and Usdin, Human Mutation, 2014; Zhao et al., PLOS Genetics, 2016; PLOS Genetics, 2018; Hayward, Steinbach and Usdin, Nucleic acids research, 2020; Miller et al., PLOS Genetics, 2020), and various spinocerebellar ataxias (Bettencourt et al., Annals of Neurology, 2016).

The present methods and compositions can be used to treat subjects who have a repeat expansion disease, e.g., Huntington's disease (HD). A diagnosis of HD can be made using methods known in the art. The cause of Huntington's disease was found to be a CAG expansion in exon 1 of the huntingtin gene (HTT). The disease protein contains a polyglutamine expansion in the N-terminal region of the Huntingtin protein (HTT) (Ellerby, L. M. (2019) Neurotherapeutics 16:924-927). Unaffected individuals may have roughly 6-29 CAG triplets in both alleles; yet, in HD patients, the disease allele may contain 36 to hundreds of CAG triplets. As the repeat number grows, the growing polyglutamine tract produces an abnormal HD gene product (called huntingtin) with increasingly aberrant properties that causes death of brain cells controlling movement (Budworth, H. and McMurray, C. T. (2013) Methods Mol Biol. 1010:3-17). Other repeat expansion diseases can be similarly diagnosed by methods known in the art. In some embodiments, the methods and compositions described herein can be administered to a cell or subject having >30 repeats, e.g., 30-100 repeats, or >100 repeats. In some embodiments, the methods and compositions described herein methods can reduce levels of huntingtin.

In some embodiments, the subject has demonstrated signs of a repeat expansion disease, e.g., HD; in some embodiments, the subject has not yet demonstrated signs of a repeat expansion disease, e.g., HD. The methods can thus be used to ameliorate one or more symptoms of a repeat expansion disease, e.g., HD, e.g., to reduce severity of one or more symptoms; to reduce the likelihood that a subject will develop one or more symptoms of the repeat expansion disease, e.g., HD; or to slow progression or worsening of one or more symptoms of the repeat expansion disease, e.g., HD.

The methods include delivering to the cell or subject a base editor and a guide RNA directing the Cas protein to the desired sequence in the MLH3 gene, e.g., as shown in Table 1, preferably SA-A4, SA-A5, or SA-A6.

The methods can include obtaining iPSC generated from differentiated somatic cells obtained from the subject; exposing the iPSC to a base editor and guide RNA treatment as described herein; optionally promoting differentiation of the corrected cells, e.g., to neural precursor cells; and administering the cells to the subject, e.g., to the CNS (spinal cord or brain) of a subject, such as to the cortex, cerebellum, hypothalamus, substantia nigra, spinal cord, putamen, hippocampus, or other CNS regions (see, e.g., Duma et al., Molecular Biology Reports volume 46, pages 5257-5272(2019); Schweitzer et al., N Engl J Med. 2020 May 14; 382(20):1926-1932; Kim et al., Alzheimers Dement (N Y). 2015 September; 1(2): 95-102), or to one or more organs (e.g., liver, lung, heart, kidney, or gut). See, e.g., WO2022197857. Alternatively, the methods can include administering a composition as described herein to the subject, e.g., to the CNS of the subject (e.g., via ICV, cisternae magna, or intrathecall administration), to an organ (e.g., liver, lung, heart, kidney, or gut) or systemically.

CRISPR Cas Targeting of mutL Homolog 3 (MLH3) Exon 7

The present methods include using base editors comprising variants of Cas DNA binding domains, e.g., Cas9 DNA binding domains with altered PAM specificity, with gRNAs that direct the base editor to a splice acceptor site (e.g., to introduce a single base mutation at the MLH3 exon 7 splice acceptor and/or splice donor site to promote skipping of exon 7) or endonuclease domain (e.g., to convert the Aspartic Acid (D) at amino acid 1223 to an Asparagine (N)), to reduce MLH3 endonuclease activity and thus reduce expansion of nucleotide repeats in the cell.

### Base Editors SpCas9 Variants

The present methods and compositions use a base editor, e.g., a cytosine base editor (CBE) or adenine base editor (ABE), e.g., an engineered Cas9 base editor (BE) construct comprising a Cas9 DNA binding domain and a deaminase domain fused at the N or C terminus or inlaid internally, and preferably also a uracil DNA glycosylase (UGI) as a fused component (see, e.g., Komor et al., Nature. 2016 May 19; 533(7603):420-4; Nishida et al., Science. 2016 Sep. 16; 353(6305); Kim et al., Nat Biotechnol. 2017 April; 35(4):371-376; Komor et al., Sci Adv. 2017 Aug. 30; 3(8):eaao4774; Gaudelli et al., Nature. 2017 Nov. 23; 551(7681):464-471); Jeong et al., Mol Ther. 2020 Sep. 2; 28(9): 1938-1952; Chu et al., The CRISPR Journal 2021 4:2, 169-177; Carrington et al., Cells. 2020 July; 9(7): 1690; and Porto et al., Nature Reviews Drug Discovery 19:839-859 (2020)). In some embodiments, the base editor is BE4max or ABEmax, e.g., as described in Koblan et al., Nat. Biotechnol. 2018; 36:843-846 or Komor et al., Sci. Adv. 2017; 3:eaao4774. Preferably the BE comprises a Cas variant as described herein. In some embodiments, the base editor comprises a deaminase domain that has been reported to more efficiently edit cytosines located within a GC sequence context (e.g., evoAPOBECI, evoCDA, evoFERNY and FERNY) (see, e.g., Thuronyi et al., Nat. Biotechnol. 37, 1070-1079 (2019). Exemplary BEs include ABEmax, ABE7.10, or ABE8e; ABEs 0.1, 0.2, 1.1, 1.2, 2.1, 2.2, 2.3, 2.4, 2.5, 2.6, 2.7, 2.8, 2.9, 2.10, 2.11, 2.12, 3.1, 3.2, 3.3, 3.4, 3.5, 3.6, 3.7, 3.8, 4.1, 4.2, 4.3, 5.1, 5.2, 5.3, 5.4, 5.5, 5.6, 5.7, 5.8, 5.9, 5.10, 5.11, 5.12, 5.13, 5.14, 6.1, 6.2, 6.3, 6.4, 6.5, 6.6, 7.1, 7.2, 7.3, 7.4, 7.5, 7.6, 7.7, 7.8, 7.9, 7.10, ABE8.8, ABE8.13, ABE8.17, ABE8.20, K20A/R21A, V82G, or V106W variants of ABE8e, miniABEmax-V82G, miniABEmax-K20A/R21A (SEQ ID 224), or miniABEmax-V106W. See, e.g., PCT/US2020/048825.

In some embodiments, the present methods and compositions use Cas DNA binding domain comprising an SpCas9 wild type or variant. In some embodiments, the Cas9/nCas9/dCas9 has altered PAM specificity, e.g., SpG (mutations at D1135L/S1136W/G1218K/E1219Q/R1335Q/T1337R, which targets NGN PAM sequences), SpRY (D1135L/S1136W/G1218K/E1219Q/R1335Q/T1337R/L1111R/A1322R/A61R/N1317R/R1333P mutations, which targets almost all PAM sequences (e.g., NRN and to a lesser extent NYN PAMs) (Walton et al., Science 26 Mar. 2020:eaba8853; WO 2021151085); VRQR (which targets NGA PAM sequences, Kleinstiver et al. Nature 523, 481-485, doi: 10.1038/nature14592 (2015); SpNRCH, SpNRRH, SpNRTH (Newby et al. Nature. 2021 July; 595(7866): 295-302; Miller et al., Nat Biotechnol. 2020 April; 38(4): 471-481); SpNG (Nishimasu et al. Science. 2018); and SpTH PAM variants. See, e.g., Jeoung et al., Scientific Reports volume 9, Article number: 4939 (2019). In some embodiments, the DNA binding domain is from SpCas9 and comprises a mutation at D1135E (NGG PAM); mutations at D1135V, R1335Q and T1337R (NGAN or NGNG PAM); mutations at D1135V, G1218R, R1335Q and T1337R (NGAN or NGNG PAM); mutations at D1135E, R1335Q and T1337R (NGAG PAM); mutations at D1135V, G1218R, R1335E and T1337R (NGCG PAM).

The SpCas9 proteins preferably include mutations at one of the following amino acid positions to create a nickase (or destroy the nuclease activity of the Cas9): D10, E762, D839, H983, or D986 and H840 or N863, e.g., D10A/D10N and H840A/H840N/H840Y, to render the nuclease portion of the protein catalytically inactive; substitutions at these positions could be alanine (as they are in Nishimasu al., Cell 156, 935-949 (2014)), or other residues, e.g., glutamine, asparagine, tyrosine, serine, or aspartate, e.g., E762Q, H983N, H983Y, D986N, N863D, N863S, or N863H (see WO 2014/152432). In some embodiments, the variant includes mutations at D10 or H840 (which creates a single-strand nickase, nCas9), or mutations at D10 and H840 (which abrogates nuclease activity; this mutant is known as dead Cas9 or dCas9).

### Guide RNAs

The methods can include the delivery of a base editor and guide RNA (gRNA) (optionally, e.g., in a ribonucleoprotein complex, or as a nucleic acid encoding the base editor and/or one or more gRNAs) bearing various spacer sequences that target the BE to the MLH3 gene. A number of exemplary Cas enzyme target sites and corresponding gRNA spacer sequences are provided in FIG. 2 and Table 1.

For the gRNA spacer sequences in Table 1, exemplary spacer sequences are shown; the 5′ end of the spacer may be extended or substituted to include alternate nucleotide compositions to modify transcription from polIII promoters. The spacer sequence can be, e.g., 20 nucleotides (nt) with a matched 5′ guanine (G), 20 nt with a mismatched 5′G, 20 nt with matched or mismatched alternate nts, 21 nt with an extended matched or mismatched 5′G or other nts, or 22 nt with extended matched or mismatched nts optionally including a 5′G. In Table 1, the gRNAs have exemplary 20 nt spacer sequences; the spacer sequences can be truncated to 19, 18, or 17 nt (by removing 1, 2, or 3 bases from the 3′ PAM distal end of the spacer).

TABLE 1

| | sgRNA spacer sequence | DNA Target Sequence | | Base | Cas |
|---|---|---|---|---|---|
| sgRNA name | (5′-3′) | (5′-3′) | PAM | Editor | protein |

MLH3 exon7 Endonuclease domain (D1223N mutation)

| | | | | | |
|---|---|---|---|---|---|
| MLH3ex7- | GATCCACCAGCACGAGCAGG | GATCCACCAGCACGAGCAGG | TTC | CBE | SpRY |
| DN-C1 | | | | | |
| MLH3ex7- | GGATCCACCAGCACGAGCAG | TGATCCACCAGCACGAGCAG | GTT | CBE | SpRY |
| DN-C2 | | | | | |
| MLH3ex7- | GTGATCCACCAGCACGAGCA | CTGATCCACCAGCACGAGCA | GGT | CBE | SpG, |
| DN-C3 | | | | | SpRY, |
| | | | | | NG, |
| | | | | | NRTH |
| MLH3ex7- | GCTGATCCACCAGCACGAGC | GCTGATCCACCAGCACGAGC | AGG | CBE | WT, |
| DN-C4 | | | | | SpG, |
| | | | | | SpRY, |
| | | | | | NG |
| MLH3ex7- | GGCTGATCCACCAGCACGAG | TGCTGATCCACCAGCACGAG | CAG | CBE | SpRY |
| DN-C5 | | | | | |

MLH3 exon7 Splice Acceptor

| | | | | | |
|---|---|---|---|---|---|
| MLH3ex7-SA- | GCTAGGTGGGAACCTGCTCG | TCTAGGTGGGAACCTGCTCG | TGC | ABE | SpG, |
| A4 | | | | | SpRY, |
| | | | | | NG, |
| | | | | | NRCH, |
| | | | | | NRTH, |
| | | | | | NRRH |
| MLH3ex7-SA- | GTCTAGGTGGGAACCTGCTC | ATCTAGGTGGGAACCTGCTC | GTG | ABE | SpG, |
| A5 | | | | | SpRY, |
| | | | | | NG, |
| | | | | | NRCH, |
| | | | | | NRTH, |
| | | | | | NRRH |
| MLH3ex7-SA- | GATCTAGGTGGGAACCTGCT | CATCTAGGTGGGAACCTGCT | CGT | ABE | SpG, |
| A6 | | | | | SpRY, |
| | | | | | NG, |
| | | | | | NRCH, |
| | | | | | NRTH, |
| | | | | | NRRH |
| MLH3ex7-SA- | GCATCTAGGTGGGAACCTGC | TCATCTAGGTGGGAACCTGC | TCG | ABE | SpRY, |
| A7 | | | | | NG |
| MLH3ex7-SA- | GTCATCTAGGTGGGAACCTG | CTCATCTAGGTGGGAACCTG | CTC | ABE | SpRY, |
| A8 | | | | | NG |
| MLH3ex7-SA- | GCTCATCTAGGTGGGAACCT | GCTCATCTAGGTGGGAACCT | GCT | ABE | SpRY |
| A9 | | | | | |
| MLH3ex7-SA- | GGCTCATCTAGGTGGGAACC | TGCTCATCTAGGTGGGAACC | TGC | ABE | SpG, |
| A10 | | | | | SpRY, |
| | | | | | NG, |
| | | | | | NRCH |
| MLH3ex7-SA- | GACCTAGATGAGCAAGGATT | CACCTAGATGAGCAAGGATT | GTG | CBE | SpRY |
| C4 | | | | | |
| MLH3ex7-SA- | GCACCTAGATGAGCAAGGAT | CCACCTAGATGAGCAAGGAT | TGT | CBE | SpG, |
| C5 | | | | | SpRY, |
| | | | | | NG |
| MLH3ex7-SA- | GCCACCTAGATGAGCAAGGA | CCCACCTAGATGAGCAAGGA | TTG | CBE | SpRY |
| C6 | | | | | |
| MLH3ex7-SA- | GCCCACCTAGATGAGCAAGG | TCCCACCTAGATGAGCAAGG | ATT | CBE | SpRY |
| C7 | | | | | |
| MLH3ex7-SA- | GTCCCACCTAGATGAGCAAG | TTCCCACCTAGATGAGCAAG | GAT | CBE | SpRY, |
| C8 | | | | | NRTH |
| MLH3ex7-SA- | GTTCCCACCTAGATGAGCAA | GTTCCCACCTAGATGAGCAA | GGA | CBE | SpG, |
| C9 | | | | | SpRY, |
| | | | | | NG, |
| | | | | | VRQR |
| MLH3ex7-SA- | GGTTCCCACCTAGATGAGCA | GGTTCCCACCTAGATGAGCA | AGG | CBE | WT, |
| C10 | | | | | SpG, |
| | | | | | SpRY, |
| | | | | | NG |

MLH3 exon7 Splice Donor

| | | | | | |
|---|---|---|---|---|---|
| MLH3ex7-SD- | GTACCAATGATAAGCTGCTC | TTACCAATGATAAGCTGCTC | CAG | ABE or | SpRY |
| A3C4 | | | | CBE | |
| MLH3ex7-SD- | GTTACCAATGATAAGCTGCT | CTTACCAATGATAAGCTGCT | CCA | ABE or | SpRY |
| A4C5 | | | | CBE | |
| MLH3ex7-SD- | GCTTACCAATGATAAGCTGC | CCTTACCAATGATAAGCTGC | TCC | ABE or | SpRY |
| A5C6 | | | | CBE | |
| MLH3ex7-SD- | GCCTTACCAATGATAAGCTG | TCCTTACCAATGATAAGCTG | CTC | ABE or | SpRY |
| A6C7 | | | | CBE | |
| MLH3ex7-SD- | GTCCTTACCAATGATAAGCT | ATCCTTACCAATGATAAGCT | GCT | ABE or | SpRY |
| A7C8 | | | | CBE | |
| MLH3ex7-SD- | GATCCTTACCAATGATAAGC | GATCCTTACCAATGATAAGC | TGC | ABE or | SpG, |
| A8C9 | | | | CBE | SpRY, |
| | | | | | NG, |
| | | | | | NRCH |
| MLH3ex7-SD- | GGATCCTTACCAATGATAAG | AGATCCTTACCAATGATAAG | CTG | ABE or | SpRY |
| A9C10 | | | | CBE | |

### Delivery and Expression Systems

The methods can include delivering the base editor (BE) and/or gRNA in a nucleic acid that encodes them. This can be performed in a variety of ways. For example, the nucleic acid encoding the BE can be delivered as mRNA or can be cloned into an intermediate vector for transformation into prokaryotic or eukaryotic cells for replication and/or expression. Intermediate vectors are typically prokaryote vectors, e.g., plasmids, or shuttle vectors, or insect vectors, for storage or manipulation of the nucleic acid encoding the BE for production of the BE. The nucleic acid encoding the BE can also be cloned into an expression vector, for administration to an animal cell, preferably a mammalian cell or a human cell, or to a fungal cell, bacterial cell, or protozoan cell.

To obtain expression, a sequence encoding a BE is typically subcloned into an expression vector that contains a promoter to direct transcription. Suitable bacterial and eukaryotic promoters are well known in the art and described, e.g., in Sambrook et al., Molecular Cloning, A Laboratory Manual (3d ed. 2001); Kriegler, Gene Transfer and Expression: A Laboratory Manual (1990); and Current Protocols in Molecular Biology (Ausubel et al., eds., 2010). Bacterial expression systems for expressing the engineered protein are available in, e.g., E. coli, Bacillus sp., and Salmonella (Palva et al., 1983, Gene 22:229-235). Kits for such expression systems are commercially available. Eukaryotic expression systems for mammalian cells, yeast, and insect cells are well known in the art and are also commercially available.

The promoter used to direct expression of a nucleic acid depends on the particular application. For example, a strong constitutive promoter is typically used for expression and purification of proteins. In contrast, when the BE is to be administered in vivo for gene regulation, either a constitutive or an inducible promoter can be used, depending on the particular use of the BE. In addition, a preferred promoter for administration of the BE can be a weak promoter, such as HSV TK or a promoter having similar activity. The promoter can also include elements that are responsive to transactivation, e.g., hypoxia response elements, Gal4 response elements, lac repressor response element, and small molecule control systems such as tetracycline-regulated systems and the RU-486 system (see, e.g., Gossen & Bujard, 1992, Proc. Natl. Acad. Sci. USA, 89:5547; Oligino et al., 1998, Gene Ther., 5:491-496; Wang et al., 1997, Gene Ther., 4:432-441; Neering et al., 1996, Blood, 88:1147-55; and Rendahl et al., 1998, Nat. Biotechnol., 16:757-761).

In addition to the promoter, the expression vector typically contains a transcription unit or expression cassette that contains all the additional elements required for the expression of the nucleic acid in host cells, either prokaryotic or eukaryotic. A typical expression cassette thus contains a promoter operably linked, e.g., to the nucleic acid sequence encoding the BE, and any signals required, e.g., for efficient polyadenylation of the transcript, transcriptional termination, ribosome binding sites, or translation termination. Additional elements of the cassette may include, e.g., enhancers, and heterologous spliced intronic signals.

The particular expression vector used to transport the genetic information into the cell is selected with regard to the intended use of the BE, e.g., expression in plants, animals, bacteria, fungus, protozoa, etc. Standard bacterial expression vectors include plasmids such as pBR322 based plasmids, pSKF, pET23D, and commercially available tag-fusion expression systems such as GST and LacZ.

Expression vectors containing regulatory elements from eukaryotic viruses are often used in eukaryotic expression vectors, e.g., lentiviral vectors, adenoviral vectors, SV40 vectors, papilloma virus vectors, and vectors derived from Epstein-Barr virus. Other exemplary eukaryotic vectors include pMSG, pAV009/A+, pMTO10/A+, pMAMneo-5, baculovirus pDSVE, and any other vector allowing expression of proteins under the direction of the SV40 early promoter, SV40 late promoter, metallothionein promoter, murine mammary tumor virus promoter, Rous sarcoma virus promoter, polyhedrin promoter, or other promoters shown effective for expression in eukaryotic cells.

Vectors for expressing the BE, guide RNAs and/or crRNAs can include RNA Pol III promoters, e.g., the H1, U6 or 7SK promoters. These human promoters allow for expression of BE, guide RNAs and/or crRNAs in mammalian cells following plasmid transfection.

Some expression systems have markers for selection of stably transfected cell lines such as thymidine kinase, hygromycin B phosphotransferase, and dihydrofolate reductase. High yield expression systems are also suitable, such as using a baculovirus vector in insect cells, with the gRNA encoding sequence under the direction of the polyhedrin promoter or other strong baculovirus promoters.

The elements that are typically included in expression vectors also include a replicon that functions in E. coli, a gene encoding antibiotic resistance to permit selection of bacteria that harbor recombinant plasmids, and unique restriction sites in nonessential regions of the plasmid to allow insertion of recombinant sequences.

Standard transfection methods are used to produce bacterial, mammalian, yeast or insect cell lines that express large quantities of protein, which are then purified using standard techniques (see, e.g., Colley et al., 1989, J. Biol. Chem., 264:17619-22; Guide to Protein Purification, in Methods in Enzymology, vol. 182 (Deutscher, ed., 1990)). Transformation of eukaryotic and prokaryotic cells are performed according to standard techniques (see, e.g., Morrison, 1977, J. Bacteriol. 132:349-351; Clark-Curtiss & Curtiss, Methods in Enzymology 101:347-362 (Wu et al., eds, 1983).

Any of the known procedures for introducing foreign nucleotide sequences into host cells may be used. These include the use of calcium phosphate transfection, polybrene, protoplast fusion, electroporation, nucleofection, liposomes, microinjection, naked DNA, plasmid vectors, viral vectors, both episomal and integrative, and any of the other well-known methods for introducing cloned genomic DNA, cDNA, synthetic DNA or other foreign genetic material into a host cell (see, e.g., Sambrook et al., supra). It is only necessary that the particular genetic engineering procedure used be capable of successfully introducing at least one gene into the host cell capable of expressing the BE.

All of the variants described herein can be rapidly incorporated into existing and widely used vectors, e.g., by simple site-directed mutagenesis.

Delivery of mRNA or AAV or other viral vectors can also be used; see, e.g., Davis et al., Nature Biomedical Engineering 6:1272-1283 (2022). In some embodiments, the BE is split into two parts to facilitate delivery in an AAV, e.g., Koblan et al. Nature 589, 608-614 (2021); Villiger et al., Nat. Med. 24, 1519-1525 (2018); Lim et al., Mol. Ther. 28, 1177-1189 (2020); She et al., Sig Transduct Target Ther 8, 57 (2023).

Adeno-associated virus is a naturally occurring defective virus that requires another virus, such as an adenovirus or a herpes virus, as a helper virus for efficient replication and a productive life cycle. (Muzyczka, Curr Top Microbiol Immunol, 158:97-129 (1992)). AAV vectors efficiently transduce various cell types and can produce long-term expression of transgenes in vivo. AAV vectors have been extensively used for gene augmentation or replacement and have shown therapeutic efficacy in a range of animal models as well as in the clinic; see, e.g., Mingozzi and High, Nat Rev Genet, 2011. 12(5): p. 341-55; Deyle and Russell, Curr Opin Mol Ther, 2009. 11(4): p. 442-7; Asokan et al., Mol Ther, 2012. 20(4): p. 699-708). AAV vectors containing as little as 300 base pairs of AAV can be packaged and can produce recombinant protein expression. In some embodiments, the AAV vector can include (or include a sequence encoding) an AAV capsid polypeptide described in PCT/US2014/060163. In some embodiments, the AAV incorporates inverted terminal repeats (ITRs). The AAV can also encode the gRNA, e.g., driven by a promoter known in the art. In some embodiments, a polymerase III promoter, such as a human U6 promoter. In some instances, the AAV vector is selected from the group consisting of AAV1, AAV2, AAV3, AAV4, AAV5, AAV6, AAV7, AAV8, AAV9, AAVrh10, AAV11, and AAV12. In some embodiments, AAV5 is used.

The AAV genomes can be packaged into AAV capsids, which capsids can be included in compositions (such as pharmaceutical compositions) and/or administered to subjects. An exemplary pharmaceutical composition comprising an AAV capsid according to this disclosure can include a pharmaceutically acceptable carrier such as balanced saline solution (BSS) and one or more surfactants (e.g., Tween 20) and/or a thermosensitive or reverse-thermosensitive polymer (e.g., pluronic). Other pharmaceutical formulation elements known in the art may also be suitable for use in the compositions described here.

An AAV vector as described herein can be a pseudotyped vector. Pseudotyping provides a mechanism for modulating a vector's target cell population. For instance, pseudotyped AAV vectors can be utilized in various methods described herein. Pseudotyped vectors are those that contain the genome of one vector, e.g., the genome of one AAV serotype, in the capsid of a second vector, e.g., a second AAV serotype. Methods of pseudotyping are well known in the art. For instance, a vector may be pseudotyped with envelope glycoproteins derived from Rhabdovirus vesicular stomatitis virus (VSV) serotypes (e.g., Indiana and Chandipura strains), rabies virus (e.g., various Evelyn-Rokitnicki-Abelseth ERA strains and challenge virus standard (CVS)), Lyssavirus Mokola virus, a rabies-related virus, vesicular stomatitis virus (VSV), Mokola virus (MV), lymphocytic choriomeningitis virus (LCMV), rabies virus glycoprotein (RV-G), glycoprotein B type (FuG-B), a variant of FuG-B (FuG-B2) or Moloney murine leukemia virus (MuLV). A virus may be pseudotyped for transduction of one or more neurons or groups of cells.

Without limitation, illustrative examples of pseudotyped vectors include recombinant AAV2/1, AAV2/2, AAV2/5, AAV2/6, AAV2/7, AAV2/8, AAV9, AAVrh10, AAV11, and AAV12 serotype vectors. It is known in the art that such vectors may be engineered to include a transgene encoding a human protein or other protein. In particular instances, the present disclosures can include a pseudotyped AAV9 or AAVrh10 viral vector including a nucleic acid as disclosed herein. See Viral Vectors for Gene Therapy: Methods and Protocols, ed. Machida, Humana Press, 2003.

In addition, the capsid can be altered to include one or more peptides that increase expression in the nervous system, e.g., in the CNS, see, e.g., Yao et al., Nat Biomed Eng. 2022 Oct. 10. doi: 10.1038/s41551-022-00938-7; Chatterjee et al., Gene Ther. 2022 June; 29(6):390-397; Meng et al., Mol Ther Methods Clin Dev. 2021 Feb. 27; 21:28-41; Zhang et al., Biomaterials. 2022 February; 281:121340; Gray, Cell Gene Ther. Insights 5, 1361-1368 (2019); Nonnenmacher et al., Mol. Ther. Methods Clin. Dev. 20, 366-378 (2021) or in the peripheral nervous system (e.g., AAV-PHP.S, Chan et al., Nat Neurosci. 2017 August; 20(8):1172-1179; AAV-MaCPNS1 and AAV-MaCPNS2, Chen et al., Neuron. 2022 Jul. 20; 110(14):2242-2257.e6; AAV-PHP, AAV-PHP.A, AAV-PHP.B, AAV-PHP.B2, AAV-PHP.B3, AAV-PHP.eB, AAV-PHP.S, Challis et al., Nat Protoc. 2019 February; 14(2):379-414). In some embodiments, the AAV vector is encapsulated by one of the following capsids: AAV1, AAV5, AAV6, AAV7, AAV8, AAV9, AAV-F, AAV-PHP, AAV-PHP.A, AAV-PHP.B, AAV-PHP.B2, AAV-PHP.B3, AAV-PHP.eB, MaCPNS1/MaCPNS2 or AAV-PHP.S.

Alternatively, the methods can include delivering the BE protein and guide RNA together, e.g., as a complex. For example, the BE and gRNA can be overexpressed in a host cell and purified, then complexed with the guide RNA (e.g., in a test tube) to form a ribonucleoprotein (RNP), and delivered to cells. In some embodiments, the BE can be expressed in and purified from bacteria through the use of bacterial BE expression plasmids. For example, His-tagged variant BE proteins can be expressed in bacterial cells and then purified using nickel affinity chromatography. The use of RNPs circumvents the necessity of delivering plasmid DNAs encoding the nuclease or the guide, or encoding the nuclease as an mRNA.

RNP delivery may also improve specificity, presumably because the half-life of the RNP is shorter and there's no persistent expression of the nuclease and guide (as you'd get from a plasmid). The RNPs can be delivered to the cells in vivo or in vitro, e.g., using lipid-mediated transfection or electroporation, extracellular vesicles, or ARRestin-domain 1 Mediated Microvesicles (ARRMs), (vesigen.com, Nabhan et al 2012 PNAS 109 (11) 4146-4151. See, e.g., Byun et al., “Gene Therapy for Huntington's Disease: The Final Strategy for a Cure?,” J Mov Disord. 2022 January; 15(1): 15-20; Liang et al. “Rapid and highly efficient mammalian cell engineering via Cas9 protein transfection.” Journal of biotechnology 208 (2015): 44-53; Zuris, John A., et al. “Cationic lipid-mediated delivery of proteins enables efficient protein-based genome editing in vitro and in vivo.” Nature biotechnology 33.1 (2015): 73-80; Kim et al. “Highly efficient RNA-guided genome editing in human cells via delivery of purified Cas9 ribonucleoproteins.” Genome research 24.6 (2014): 1012-1019.

### Compositions

Also described herein are compositions comprising a gRNA and BE, that can be administered to a subject in need thereof. The compositions can include, e.g., a viral delivery vector, e.g., preferably an adeno-associated virus (AAV) vector that comprises sequences encoding the sgRNA and BE (as noted above, the BE can be split, and encoded across two AAV). Alternatively, the compositions can comprise a RNP comprising the BE complexed with the guide RNA.

Also described herein are pharmaceutical compositions comprising or consisting of a gRNA and BE, or a nucleic acid encoding the gRNA and BE, as an active ingredient.

Pharmaceutical compositions typically include a pharmaceutically acceptable carrier. As used herein the language “pharmaceutically acceptable carrier” includes saline, solvents, dispersion media, coatings, antibacterial and antifungal agents, isotonic and absorption delaying agents, and the like, compatible with pharmaceutical administration.

Pharmaceutical compositions are typically formulated to be compatible with its intended route of administration. Examples of routes of administration include parenteral, e.g., intravenous, intradermal, subcutaneous, oral (e.g., inhalation), transdermal (topical), transmucosal, and rectal administration.

Methods of formulating suitable pharmaceutical compositions are known in the art, see, e.g., Remington: The Science and Practice of Pharmacy, 21st ed., 2005; and the books in the series Drugs and the Pharmaceutical Sciences: a Series of Textbooks and Monographs (Dekker, NY). For example, solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use can include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, NJ) or phosphate buffered saline (PBS). In all cases, the composition must be sterile and should be fluid to the extent that easy syringability exists. It should be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, sodium chloride in the composition. Prolonged absorption of the injectable compositions can be brought about by including in the composition an agent that delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle, which contains a basic dispersion medium and the required other ingredients from those enumerated above. In the case of sterile powders for the preparation of sterile injectable solutions, the preferred methods of preparation are vacuum drying and freeze-drying, which yield a powder of the active ingredient plus any additional desired ingredient from a previously sterile-filtered solution thereof.

Oral compositions generally include an inert diluent or an edible carrier. For the purpose of oral therapeutic administration, the active compound can be incorporated with excipients and used in the form of tablets, troches, or capsules, e.g., gelatin capsules. Oral compositions can also be prepared using a fluid carrier for use as a mouthwash. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds can be delivered in the form of an aerosol spray from a pressured container or dispenser that contains a suitable propellant, e.g., a gas such as carbon dioxide, or a nebulizer. Such methods include those described in U.S. Pat. No. 6,468,798.

Systemic administration of a therapeutic compound as described herein can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The pharmaceutical compositions can also be prepared in the form of suppositories (e.g., with conventional suppository bases such as cocoa butter and other glycerides) or retention enemas for rectal delivery.

Therapeutic compounds that are or include nucleic acids can be administered by any method suitable for administration of nucleic acid agents, such as a DNA vaccine. These methods include gene guns, bio injectors, and skin patches as well as needle-free methods such as the micro-particle DNA vaccine technology disclosed in U.S. Pat. No. 6,194,389, and the mammalian transdermal needle-free vaccination with powder-form vaccine as disclosed in U.S. Pat. No. 6,168,587. Additionally, intranasal delivery is possible, as described in, inter alia, Hamajima et al., Clin. Immunol. Immunopathol., 88(2), 205-10 (1998). Liposomes (e.g., as described in U.S. Pat. No. 6,472,375) and microencapsulation can also be used. Biodegradable targetable microparticle delivery systems can also be used (e.g., as described in U.S. Pat. No. 6,471,996).

In some embodiments, the therapeutic compounds are prepared with carriers that will protect the therapeutic compounds against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Such formulations can be prepared using standard techniques, or obtained commercially, e.g., from Alza Corporation and Nova Pharmaceuticals, Inc. Liposomal suspensions (including liposomes targeted to selected cells with monoclonal antibodies to cellular antigens) can also be used as pharmaceutically acceptable carriers. These can be prepared according to methods known to those skilled in the art, for example, as described in U.S. Pat. No. 4,522,811.

The pharmaceutical compositions can be included in a kit, container, pack, or dispenser together with instructions for administration.

### EXAMPLES

The invention is further described in the following examples, which do not limit the scope of the invention described in the claims.

### Example 1

Guide RNA spacer sequences with 20 nt were designed to tile the MLH3 exon 7 splice acceptor motif (FIG. 2). Spacer sequences were cloned into BsbBI sites in plasmid BPK1520 (Addgene #65777), which is a human expression plasmid for SpCas9 sgRNA (Kleinstiver et al Nature. 2015). The following ABE/CBE expression plasmids were tested: ABE8e-SpWT (Addgene #185910), ABE8e-SpRY (Addgene #185912), ABE8e-SpG (Addgene #185911), ABE8.20m-SpWT (Addgene #185915), ABE8.20m-SpRY (Addgene #185917), ABE8.20m-SpG (Addgene #185916), CBE4max-SpWT, CBE4max-SpRY (Addgene #139999), CBE4max-SpG (Addgene #139998) (Walton et al Science. 2020, Alves et al bioRxiv. 2023). Efficiency of base editing was determined in HEK293T cells: on day 1, cells were plated in 24-well plates (~2×10⁵ cells/well); on day 2, cells were transfected using Lipofectamine 3000 with combinations of a ABE/CBE expression plasmid (~350 ng) and a sgRNA expression plasmid (~150 ng); cells were imaged for GFP to confirm transfection efficiency and passaged as needed; on day 8, cells were collected for DNA and RNA molecular analysis. For accurate quantification of base editing efficiency, next generation sequencing analysis was performed. Primers were designed to flank the human MLH3 exon 7 (forward primer: 5′-ACACACATGATGGTTGTCGTCT; reverse primer: 5′-GCTTTCTCACAACAGTTGCAGTT) and generate a 228 bp PCR product. Using these primers, PCR products were generated using DNA obtained from each independent base editing combination. PCR products were barcoded (8 nt) and sequenced using Illumina sequencing with 140 bp paired-end reads. Base editing efficiencies were determined using CRISPRessov2 (Clement et al Nat. Biotechnol. 2019) using a minimum of 5,000 reads per sample. Results obtained are summarized in FIG. 3A. Treatment with CBE plus guide resulted in editing of splice acceptor motif (i.e. GCTCATCTAG->GCTCATCTAA) in ~20% of alleles. Treatment with ABE plus guide resulted in editing of splice acceptor motif (i.e. GCTCATCTAG->GCTCATCTCG) in ~40% of alleles: the combinations (BE:sgRNA) of ABE-SpG:SA-A6 (~43%), ABE-SpG:SA-A4 (~40%), and ABE-SpRY:SA-A5 (~29%) resulted in the highest editing efficiencies, respectively (FIG. 3A).

Single cell clones were isolated from ABE8e-SpG plus A6 sgRNA treated cells. Editing at the MLH3 exon 7 splice acceptor motif was validated by Sanger sequencing. RNA was extracted from clones that were 100% unedited (i.e. A/A homozygous), 50% edited (i.e. A/G heterozygous) and 100% edited (i.e. G/G homozygous). RNA was converted to cDNA using SuperScript III First-Strand Synthesis System (ThermoFisher). RT-PCR was performed using primers hMLH3iso-F3: 5′-CCAGCAAGTAGATAACAAGTTTATTGC and hMLH3iso-R3: 5′-CTCTAGCGGAGGAATTAGAGTAGAA. MLH3 isoform 1 (containing endonuclease domain) was the predominant variant detected in 100% unedited clones (FIG. 3B). MLH3 isoform 1 was significantly reduced in 50% edited cells, with the isoform 2 being the predominant MLH3 variant detected. In 100% edited cells, MLH3 isoform 1 was undetected and only MLH3 isoform 2 (missing endonuclease domain) was detected (FIG. 3B). This demonstrates that a single base change in the MLH3 exon 7 splice acceptor motif (eg. GCTCATCTAG->GCTCATCTCG) causes complete splice exclusion of the endonuclease-containing MLH3 exon 7.

### Example 2

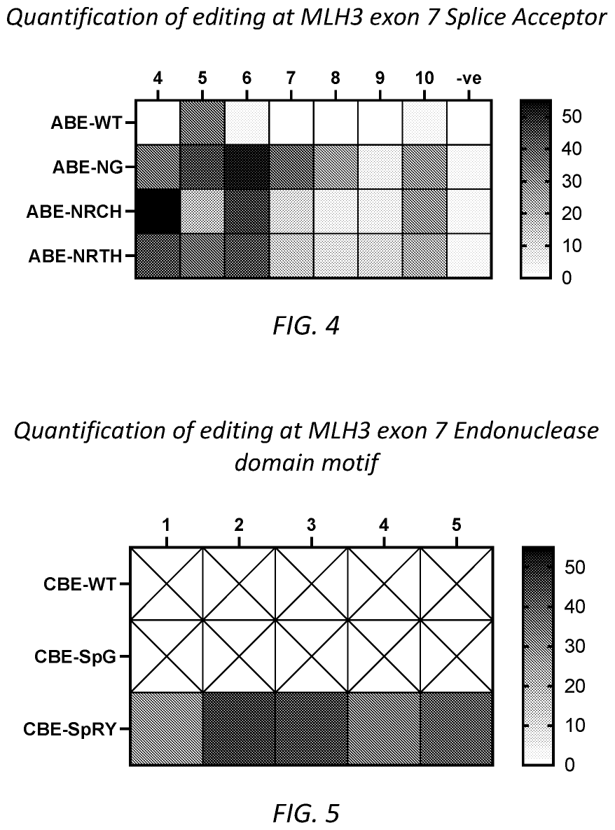

Guide RNA spacer sequences with 20 nt were designed to tile the MLH3 exon 7 splice acceptor motif (FIG. 2). Spacer sequences were cloned into BsbBI sites in plasmid BPK1520 (Addgene #65777), which is a human expression plasmid for SpCas9 sgRNA (Kleinstiver et al Nature. 2015). The following ABE expression plasmids were tested: ABE8e-SpWT (Addgene #185910), ABE8e-SpNG, ABE8e-SpNRCH, and ABE8e-SpNRTH (Walton et al Science. 2020, Alves et al bioRxiv. 2023; Arbab et al Science. 2023; Miller et al. Nat Biotechnol. 2020). Efficiency of base editing was determined in HEK293T cells: on day 1, cells were plated in 96-well plates (~5×10⁴ cells/well); on day 2, cells were transfected using Lipofectamine 3000 with combinations of a ABE expression plasmid (~85 ng) and a sgRNA expression plasmid (~35 ng); cells were imaged for GFP to confirm transfection efficiency and passaged as needed; on day 4, cells were collected for DNA and RNA molecular analysis. For accurate quantification of base editing efficiency, next generation sequencing analysis was performed. Primers were designed to flank the human MLH3 exon 7 (forward primer: 5′-ACACACATGATGGTTGTCGTCT; reverse primer: 5′-GCTTTCTCACAACAGTTGCAGTT) and generate a 228 bp PCR product. Using these primers, PCR products were generated using DNA obtained from each independent base editing combination. PCR products were barcoded (8 nt) and sequenced using Illumina sequencing with 140 bp paired-end reads. Base editing efficiencies were determined using CRISPRessov2 (Clement et al Nat. Biotechnol. 2019) using a minimum of 5,000 reads per sample. Results obtained are summarized in FIG. 4. Treatment with ABE plus guide resulted in editing of splice acceptor motif (i.e. GCTCATCTAG->GCTCATCTCG) in ~55% of alleles: the combinations (BE:sgRNA) of ABE-SpNRCH:SA-A4 (~55%), ABE-SpNG:SA-A6 (~52%), ABE-SpNRCH:SA-A6 (~46%), ABE-SpNG:SA-A5 (~45%), ABE-SpNRTH:SA-A4 (~44%), and ABE-SpNRTH:SA-A6 (~43%) resulted in the highest editing efficiencies, respectively (FIG. 4).

### Example 3

Guide RNA spacer sequences with 20 nt were designed to tile the endonuclease motif in the MLH3 exon 7 (FIG. 2). Spacer sequences were cloned into BsbBI sites in plasmid BPK1520 (Addgene #65777), which is a human expression plasmid for SpCas9 sgRNA (Kleinstiver et al Nature. 2015). The following CBE expression plasmid was tested: CBE4max-SpRY (Addgene #139999) (Walton et al Science. 2020). Efficiency of base editing was determined in HEK293T cells: on day 1, cells were plated in 24-well plates (~2×10⁵ cells/well); on day 2, cells were transfected using Lipofectamine 3000 with combinations of a ABE/CBE expression plasmid (~350 ng) and a sgRNA expression plasmid (~150 ng); cells were imaged for GFP to confirm transfection efficiency and passaged as needed; on day 8, cells were collected for DNA and RNA molecular analysis. For accurate quantification of base editing efficiency, next generation sequencing analysis was performed. Primers were designed to flank the human MLH3 exon 7 (forward primer: 5′-ACACACATGATGGTTGTCGTCT; reverse primer: 5′-GCTTTCTCACAACAGTTGCAGTT) and generate a 228 bp PCR product. Using these primers, PCR products were generated using DNA obtained from each independent base editing combination. PCR products were barcoded (8 nt) and sequenced using Illumina sequencing with 140 bp paired-end reads. Base editing efficiencies were determined using CRISPRessov2 (Clement et al Nat. Biotechnol. 2019) using a minimum of 5,000 reads per sample. Results obtained are summarized in FIG. 5. Treatment with CBE plus guide resulted in editing of splice acceptor motif (i.e. GTGGATCAG->GTGAATCAG, which translates to VDQ->VNQ) in ~45% of alleles: the combinations (BE:sgRNA) of CBE-SpRY:DN-C2 (~47%), CBE-SpRY:DN-C3 (~44%), and CBE-SpRY:DN-C5 (~41%) resulted in the highest editing efficiencies, respectively (FIG. 5).

### Example 4. Testing of MLH3 Splice Modulation with Candidate CRISPR Base Editors in an HD Mouse Model

Selected base editors (top ABE:sgRNA pairs, FIGS. 3A and 4) are tested against the MLH3 exon 7 splice acceptor. Since the MLH3-targeting BEs were designed against the human sequence, and the MLH3-SA genomic sequence does not have perfect homology between mouse and human, we generated a humanized Mlh3 transgenic mouse, where we replaced the mouse exon containing the endonuclease, plus 30-40 bp of flanking region on either end, with the human corresponding sequence. We have completed validation of this transgenic line and we are crossing it with Htt.Q111, to generate HD mice homozygous for the humanized Mlh3 transgene for use in the proposed experiments. We will package BEs into AAV capsids and deliver them either by neonate (P0-P2) bilateral ICV injection (e.g., using AAV9) or by tail vein injection at 6 weeks of age (using PHP.eB), with either total 1E+11VG (concentrated in 2 ul) or 3E+12VG (in 200 ul) per mouse. A total of 6 animals will be injected per condition. We will also inject control animals with PBS and/or empty vector capsids. It is worth noting that since the base editors are larger than the typical capacity of AAV vectors, this will be packaged and delivered as two AAVs using a “split intein” strategy (Levy et al Nat Biomed Eng. 2020). We will age animals up to 24 weeks of age, which is a timepoint we use in other HD studies, since at this age the animals display significant somatic CAG expansions in the striatum and liver tissues. We will dissect brain (eg. striatum, cortex, cerebellum, brain stem) and other peripheral tissues (eg. liver, heart) and perform detailed characterization of MLH3 editing, as well as quantification of somatic CAG expansions using fragment analysis, as well as long-read sequencing methods. Importantly, we will co-inject a KASH-GFP virus that will allow for FACS sorting of successfully transduced nuclei, and therefore more accurate quantification of editing in cells that are actually exposed to the ABEs. We will also perform histological analysis to demonstrate that treatment rescued HD phenotypes such as mutant HTT accumulation in the nucleus of medium spiny neurons (i.e., EM48). AAV-based delivery will be prioritized, especially being cognizant of constant developments in the field of AAV engineering with many novel CNS specific capsids capable of crossing the BBB. Alternative non-viral delivery strategies that may have advantageous safety profiles and also allow for repeated dosing, will also be explored if necessary, including extracellular vesicles (EVs) such as ARRDC1-mediated microvesicles (ARMMs), for non-viral delivery of therapeutic payloads. ARMMs are naturally occurring vesicles that mediate intercellular communication across different tissues. Their biogenesis is driven by a protein called ARRDC1 working in concert with other cellular factors. Briefly, ARRDC1 is utilized as an active recruitment handle to intraluminally load RNP in ARMMs. Production and purification of RNP-loaded ARMMs is scalable and can readily support clinical applications. ARMMs loaded with the candidate ABE base editor protein complexed with the candidate MLH3-targeting gRNA will be used for intra-cranial evaluation in Htt.Q111 mice that are homozygous for the humanized Mlh3 transgene, as a non-viral therapeutic strategy that can ultimately be implemented in patients.

### Example 5. Testing of MLH3 Splice Modulation with Candidate Base Editors in a Human Cellular Model of CAG Expansions

Selected ABE base editors for MLH3 (ABE:sgRNA pairs) will be tested in a human HTT CAG cell line (RPE1-AAVS1-CAG115), developed for modeling somatic expansions (McLean et al. bioRxiv 2023.07.25.550489; doi.org/10.1101/2023.07.25.550489). This will primarily be aimed at validating that the candidate reagents are not only effective at introducing base edits at the MLH3 target site, but also, consequently capable of preventing HTT CAG expansions in a human system. Most cultured HD cell lines display limited CAG repeat instability, and the ones that do, are slow and rely on active cell division, which may not be representative of mechanisms driving somatic CAG expansions in non-dividing neurons, such as medium spiny neurons affected in HD patient's brains. To address this unmet need, we have developed a new model system for this purpose in hTERT-RPE1 (RPE1) cells. RPE1 is a near-diploid immortalized cell line often used to study DNA repair pathways (Olivieri and Durocher, STAR Protoc. 2021 Mar. 19; 2(1): 100321). It can be arrested at GO/1 through contact inhibition by growing the cells to confluency. We isolated the expanded CAG HTT exon 1 from a juvenile-onset HD individual (115 CAGs) and knocked the fragment into the AAVS1 safe harbor locus (intron 1 of PPP1R12C on chromosome 19). Isolated clones showed relatively rapid CAG repeat expansion arrested at GO/1 with an average CAG weekly gain of 1.34 (95% CI: 1.22-1.47) (McLean et al. bioRxiv 2023.07.25.550489; doi.org/10.1101/2023.07.25.550489). We validated the relevance of the RPE1-AAVS1-CAG115 cell line to model somatic instability processes by perturbing modifiers of HD age-at-onset predicted to influence repeat instability. We utilized CRISPR-Cas9 nuclease to target and modify the coding sequences of FAN1, MLH3, MSH3, and PMS1 via loss-of-function indel mutations and analyzed repeat instability (30-80% editing; bulk measurement by NGS). As expected, FAN1 knockout increased the average CAG repeat gain per week from 1.34 to 2.52 (p<0.0001). By contrast, knockout of MLH3, MSH3 and PMS1 completely prevented expansion of CAG repeats (p<0.0001) (McLean et al bioRxiv (Preprint). 2023)., further supporting the goal of this proposal in pursuing these genetic modifiers as therapeutic targets.

Additionally, we will also confirm that the candidate ABE and sgRNA pairs, that maximize on-target editing of MLH3 with minimal bystander edits, are not causing any detrimental side effects by testing for genome-wide off-target edits using unbiased methods, such as CHANGE-seq, and performing RNA-seq to detect any eventual transcriptomic alterations. In summary, to monitor Cas9 or gRNA-dependent DNA off-targets, we will conduct in silico analyses using CasOFFinder to nominate genomic sites that are similar in sequence to the on-target site (Bae 2014), will perform unbiased genome-wide cell-based off-target assays such as GUIDE-seq (Tsai 2014), and also comprehensive biochemical assays that can be modified to accommodate the use of base editors, such as CHANGE-seq (Lazzarotto 2020). These assays will permit us to nominate potential off-target sites and thus determine whether editing is specific to the MLH3 locus. Using genomic DNA from our cell-based models treated with the ABE/gRNA plasmids, we will then deeply sequence these off-target sites via NGS. Should we identify off-targets for any BE/gRNA combinations, we will engineer the base editors with our previously developed high-fidelity mutations in Cas9 that we have shown to reduce unwanted off-target editing (Kleinstiver 2016; Chen 2017; Walton 2020). In addition to DNA-based off-targets we will also monitor potential transcriptome-wide edits, which we can also minimize using previously described mutations or BE configurations that reduce the propensity of base editors to mutate RNA (Grunewald 2019, Rees 2019; Cao 2022). Finally, to account for genetic diversity between patients, we will perform off-target analysis for the candidate BE pair in three HD patient-derived fibroblast cell lines, using the same methodology described above.

### Example 6. Design and In Vitro Screen CRISPR Base/Prime Editing Reagents Against MSH3 and PMS1 Genes

Based on prior extensive work characterizing genetic modifiers of somatic CAG repeat instability, and their minimal oncogenic liability (Prolla 1998, van Oers 2013), we propose MSH3 and PMS1 as additional genetic targets. We will employ similar design strategies that we have so far used for MLH3 and test multiple combinations of base editors and/or prime editors targeting candidate regions in MSH3 and PMS1, which have previously been shown to be strong modifiers of CAG expansions both in mice and human cells. This will generate additional reagents for alternative therapeutic targets, which will not only allow us to mitigate risk of focusing solely on MLH3, while it may actually result in reagents with more favorable efficiency and safety profiles.

As we have already done for MLH3, we will primarily focus on targeting splicing sites in both MSH3 and PMS1 that result in switching expression from the canonical full isoforms, to naturally occurring isoforms that do not cause CAG expansions. In parallel, we will also prioritize the targeting of two sites responsible for MSH3's ATPase activity—Walker A and Walker B domains (Kumar 2013, Keogh 2017)—for which we have previously shown that a single base change in either site results in complete suppression of somatic CAG expansion in the striatum of Htt.Q111 mice, while still resulting in the expression of an MSH3 protein. This approach will be limited to MSH3 since for PMS1 no functional domains have been previously identified, which may explain its apparently dispensary role in canonical mismatch repair (reviewed in Iyer and Pluciennik 2021). However, given that we are trying to generate loss-of-function variants of both MSH3 and PMS1, we will also consider introducing missense/nonsense mutations in the most editing amenable sites (based on CRISPR/Cas9 on- and off-target predictions), that will ultimately have the same impact on somatic CAG expansions. We will design base (ABE/CBE) and/or prime (PE) editors by tilling all possible sgRNAs for selected regions of interest and testing them in combination with multiple Cas9 PAM variants. This high-throughput screen will be performed in the RPE1-AAVS1-CAG115 cell line, which will simultaneously allow for evaluation of editing efficiency and confirmation of impact on HTT CAG expansions. Essentially, cells will be plated in 96 well plates and transfected with unique combinations of plasmids expressing sgRNA of interest and ABE/CBE/PE (each unique combination will be tested in two separate replica plates and triplicates per plate; untreated and empty vector controls will be included as well). Antibiotic selection will be applied for 72 hrs to enrich for successfully transfected cell. At this point, one of the replica plates will be harvested for DNA extraction and downstream evaluation of editing efficiency by next generation sequencing and CRISPResso2 bioinformatic analysis (Clement 2029). The cells in the other replica plate will then be grown to confluence, consequently stopping cell replication. After 21 days (if successful editing is observed in the first replica plate), cells will be harvested for DNA and CAG expansions will be sized by fragment analysis. After only 21 days in culture, untreated and empty control cells are expected to gain ~4 CAG units, providing sufficient increase in CAG length to detect significant stabilization effects from best performing gene editing reagents. The top 5 candidate BE/PE reagents identified for each gene will be repeated for validation of results, as well as for RNA and protein quantification of MSH3/PMS1. Off-target analysis will be performed for the best performing candidates.

### REFERENCES

Alves, C. R. R., Ha, L. L., Yaworski, R., Lazzarotto, C. R., Christie, K. A., Reilly, A., Beauvais, A., Doll, R. M., de la Cruz, D., Maguire, C. A., Swoboda, K. J., Tsai, S. Q., Kothary, R., & Kleinstiver, B. P. (2023). Base editing as a genetic treatment for spinal muscular atrophy. BioRxiv: The Preprint Server for Biology. doi.org/10.1101/2023.01.20.524978
Ament, S. A., Pearl, J. R., Grindeland, A., Claire, J. S., Earls, J. C., Kovalenko, M., Gillis, T., Mysore, J., Gusella, J. F., Lee, J. M., Kwak, S., Howland, D., Lee, M. Y., Baxter, D., Scherler, K., Wang, K., Geman, D., Carroll, J. B., MacDonald, M. E., . . . Hood, L. E. (2017). High resolution time-course mapping of early transcriptomic, molecular and cellular phenotypes in Huntington's disease CAG knock-in mice across multiple genetic backgrounds. Human Molecular Genetics, 26(5), 913-922. doi.org/10.1093/HMG/DDX006
Arbab, M., Matuszek, Z., Kray, K. M., Du, A., Newby, G. A., Blatnik, A. J., Raguram, A., Richter, M. F., Zhao, K. T., Levy, J. M., Shen, M. W., Arnold, W. D., Wang, D., Xie, J., Gao, G., Burghes, A. H. M., & Liu, D. R. (2023). Base editing rescue of spinal muscular atrophy in cells and in mice. Science (New York, N.Y.), 380(6642), eadg6518. doi.org/10.1126/SCIENCE.ADG6518
Asokan, A., Schaffer, D. V., & Samulski, R. J. (2012). The AAV vector toolkit: poised at the clinical crossroads. Molecular Therapy: The Journal of the American Society of Gene Therapy, 20(4), 699-708. doi.org/10.1038/MT.2011.287
Bettencourt, C., Hensman-Moss, D., Flower, M., Wiethoff, S., Brice, A., Goizet, C., Stevanin, G., Koutsis, G., Karadima, G., Panas, M., Yescas-Gómez, P., Garcia-Velizquez, L. E., Alonso-Vilatela, M. E., Lima, M., Raposo, M., Traynor, B., Sweeney, M., Wood, N., Giunti, P., . . . Jones, L. (2016). DNA repair pathways underlie a common genetic mechanism modulating onset in polyglutamine diseases. Annals of Neurology, 79(6), 983-990. doi.org/10.1002/ANA.24656
Bourn, R. L., de Biase, I., Pinto, R. M., Sandi, C., Al-Mahdawi, S., Pook, M. A., & Bidichandani, S. I. (2012). Pms2 suppresses large expansions of the (GAA TTC)n sequence in neuronal tissues. PloS One, 7(10). doi.org/10.1371/JOURNAL.PONE.0047085
Budworth, H., & McMurray, C. T. (2013). A brief history of triplet repeat diseases. Methods in Molecular Biology (Clifton, N.J.), 1010, 3-17. doi.org/10.1007/978-1-62703-411-1_1
Cannavo, E., Marra, G., Sabates-Bellver, J., Menigatti, M., Lipkin, S. M., Fischer, F., Cejka, P., & Jiricny, J. (2005). Expression of the MutL homologue hMLH3 in human cells and its role in DNA mismatch repair. Cancer Research, 65(23), 10759-10766. doi.org/10.1158/0008-5472.CAN-05-2528
Carrington, B., Weinstein, R. N., & Sood, R. (2020). BE4max and AncBE4max Are Efficient in Germline Conversion of C:G to T:A Base Pairs in Zebrafish. Cells, 9(7). doi.org/10.3390/CELLS9071690
Charbonneau, N., Amunugama, R., Schmutte, C., Yoder, K., & Fishel, R. (2009). Evidence that hMLH3 functions primarily in meiosis and in hMSH2-hMSH3 mismatch repair. Cancer Biology & Therapy, 8(14), 1411-1420. doi.org/10.4161/CBT.8.14.8886
Chu, S. H., Packer, M., Rees, H., Lam, D., Yu, Y., Marshall, J., Cheng, L. I., Lam, D., Olins, J., Ran, F. A., Liquori, A., Gantzer, B., Decker, J., Born, D., Barrera, L., Hartigan, A., Gaudelli, N., Ciaramella, G., & Slaymaker, I. M. (2021). Rationally Designed Base Editors for Precise Editing of the Sickle Cell Disease Mutation. The CRISPR Journal, 4(2), 169-177. doi.org/10.1089/CRISPR.2020.0144
Ciosi, M., Maxwell, A., Cumming, S. A., Hensman Moss, D. J., Alshammari, A. M., Flower, M. D., Durr, A., Leavitt, B. R., Roos, R. A. C., Holmans, P., Jones, L., Langbehn, D. R., Kwak, S., Tabrizi, S. J., & Monckton, D. G. (2019). A genetic association study of glutamine-encoding DNA sequence structures, somatic CAG expansion, and DNA repair gene variants, with Huntington disease clinical outcomes. EBioMedicine, 48, 568-580. doi.org/10.1016/J.EBIOM.2019.09.020
Claeys Bouuaert, C., & Keeney, S. (2017). Distinct DNA-binding surfaces in the ATPase and linker domains of MutL7 determine its substrate specificities and exert separable functions in meiotic recombination and mismatch repair. PLoS Genetics, 13(5). doi.org/10.1371/JOURNAL.PGEN.1006722
Clark-Curtiss, J. E., & Curtiss, R. (1983). Analysis of recombinant DNA using Escherichia coli minicells. Methods in Enzymology, 101(C), 347-362. doi.org/10.1016/0076-6879(83)01026-5
Clement, K., Rees, H., Canver, M. C., Gehrke, J. M., Farouni, R., Hsu, J. Y., Cole, M. A., Liu, D. R., Joung, J. K., Bauer, D. E., & Pinello, L. (2019). CRISPResso2 provides accurate and rapid genome editing sequence analysis. Nature Biotechnology, 37(3), 224-226. doi.org/10.1038/S41587-019-0032-3
Davis, J. R., Wang, X., Witte, I. P., Huang, T. P., Levy, J. M., Raguram, A., Banskota, S., Seidah, N. G., Musunuru, K., & Liu, D. R. (2022). Efficient in vivo base editing via single adeno-associated viruses with size-optimized genomes encoding compact adenine base editors. Nature Biomedical Engineering, 6(11), 1272-1283. doi.org/10.1038/S41551-022-00911-4
Deyle, D. R., & Russell, D. W. (2009). Adeno-associated virus vector integration. Current Opinion in Molecular Therapeutics, 11(4), 442./pmc/articles/PMC2929125/
Dragileva, E., Hendricks, A., Teed, A., Gillis, T., Lopez, E. T., Friedberg, E. C., Kucherlapati, R., Edelmann, W., Lunetta, K. L., MacDonald, M. E., & Wheeler, V. C. (2009). Intergenerational and striatal CAG repeat instability in Huntington's disease knock-in mice involve different DNA repair genes. Neurobiology of Disease, 33(1), 37-47. doi.org/10.1016/J.NBD.2008.09.014
Duma, C., Kopyov, O., Kopyov, A., Berman, M., Lander, E., Elam, M., Arata, M., Weiland, D., Cannell, R., Caraway, C., Berman, S., Scord, K., Stemler, L., Chung, K., Khoudari, S., McRory, R., Duma, C., Farmer, S., Bravo, A., . . . Rapaport, B. (2019). Human intracerebroventricular (ICV) injection of autologous, non-engineered, adipose-derived stromal vascular fraction (ADSVF) for neurodegenerative disorders: results of a 3-year phase 1 study of 113 injections in 31 patients. Molecular Biology Reports, 46(5), 5257-5272. doi.org/10.1007/S11033-019-04983-5
Ellerby, L. M. (2019). Repeat Expansion Disorders: Mechanisms and Therapeutics. Neurotherapeutics: The Journal of the American Society for ExperimentalNeuroTherapeutics, 16(4), 924-927. doi.org/10.1007/S13311-019-00823-3
Ezzatizadeh, V., Sandi, C., Sandi, M., Anjomani-Virmouni, S., Al-Mahdawi, S., & Pook, M. A. (2014). MutLa heterodimers modify the molecular phenotype of Friedreich ataxia. PloS One, 9(6). doi.org/10.1371/JOURNAL.PONE.0100523
Flower, M., Lomeikaite, V., Ciosi, M., Cumming, S., Morales, F., Lo, K., Hensman Moss, D., Jones, L., Holmans, P., Monckton, D. G., Tabrizi, S. J., Kraus, P., Hoffman, R., Tobin, A., Borowsky, B., Keenan, S., Whitlock, K. B., Queller, S., Campbell, C., . . . Merkies, I. (2019). MSH3 modifies somatic instability and disease severity in Huntington's and myotonic dystrophy type 1. Brain: A Journal of Neurology, 142(7), 1876-1886. doi.org/10.1093/BRAIN/AWZ115
Foiry, L., Dong, L., Savouret, C., Hubert, L., te Riele, H., Junien, C., & Gourdon, G. (2006). Msh3 is a limiting factor in the formation of intergenerational CTG expansions in DM1 transgenic mice. Human Genetics, 119(5), 520-526. doi.org/10.1007/S00439-006-0164-7
Fox, L. M. (2006). The Science and Practice of Pharmacy, 21st Edition. American Journal of Pharmaceutical Education, 70(3). /pmc/articles/PMC1636967/
Gaudelli, N. M., Komor, A. C., Rees, H. A., Packer, M. S., Badran, A. H., Bryson, D. I., & Liu, D. R. (2017). Programmable base editing of A⋅T to G⋅C in genomic DNA without DNA cleavage. Nature, 551(7681), 464-471. doi.org/10.1038/NATURE24644
Gonitel, R., Moffitt, H., Sathasivam, K., Woodman, B., Detloff, P. J., Faull, R. L. M., & Bates, G. P. (2008). DNA instability in postmitotic neurons. Proceedings of the National Academy of Sciences of the United States of America, 105(9), 3467-3472. doi.org/10.1073/PNAS.0800048105
Gossen, M., & Bujard, H. (1992). Tight control of gene expression in mammalian cells by tetracycline-responsive promoters. Proceedings of the National Academy of Sciences of the United States of America, 89(12), 5547-5551. doi.org/10.1073/PNAS.89.12.5547
Halabi, A., Ditch, S., Wang, J., & Grabczyk, E. (2012). DNA mismatch repair complex MutSβ promotes GAA TTC repeat expansion in human cells. The Journal of Biological Chemistry, 287(35), 29958-29967. doi.org/10.1074/JBC.M112.356758
Halabi, A., Fuselier, K. T. B., & Grabczyk, E. (2018). GAA⋅TTC repeat expansion in human cells is mediated by mismatch repair complex MutL7 and depends upon the endonuclease domain in MLH3 isoform one. Nucleic Acids Research, 46(8), 4022-4032. doi.org/10.1093/NAR/GKY143
Hamajima, K., Sasaki, S., Fukushima, J., Kaneko, T., Xin, K. Q., Kudoh, I., & Okuda, K. (1998). Intranasal administration of HIV-DNA vaccine formulated with a polymer, carboxymethylcellulose, augments mucosal antibody production and cell-mediated immune response. Clinical Immunology and Immunopathology, 88(2), 205-210. doi.org/10.1006/clin.1998.4566
Hayward, B. E., Steinbach, P. J., & Usdin, K. (2020). A point mutation in the nuclease domain of MLH3 eliminates repeat expansions in a mouse stem cell model of the Fragile X-related disorders. Nucleic Acids Research, 48(14), 7856-7863. doi.org/10.1093/NAR/GKAA573
Jeong, Y. K., Song, B., & Bae, S. (2020). Current Status and Challenges of DNA Base Editing Tools. Molecular Therapy: The Journal of the American Society of Gene Therapy, 28(9), 1938-1952. doi.org/10.1016/J.YMTHE.2020.07.021
Kadyrov, F. A., Dzantiev, L., Constantin, N., & Modrich, P. (2006). Endonucleolytic function of MutLalpha in human mismatch repair. Cell, 126(2), 297-308. doi.org/10.1016/J.CELL.2006.05.039
Kadyrova, L. Y., Gujar, V., Burdett, V., Modrich, P. L., & Kadyrov, F. A. (2020). Human MutLγ, the MLH1-MLH3 heterodimer, is an endonuclease that promotes DNA expansion. Proceedings of the National Academy of Sciences of the United States of America, 117(7), 3535-3542. doi.org/10.1073/PNAS.1914718117/-/DCSUPPLEMENTAL
Kennedy, L., Evans, E., Chen, C. M., Craven, L., Detloff, P. J., Ennis, M., & Shelbourne, P. F. (2003). Dramatic tissue-specific mutation length increases are an early molecular event in Huntington disease pathogenesis. Human Molecular Genetics, 12(24), 3359-3367. doi.org/10.1093/HMG/DDG352
Kim, D., Lim, K., Kim, S. T., Yoon, S. H., Kim, K., Ryu, S. M., & Kim, J. S. (2017). Genome-wide target specificities of CRISPR RNA-guided programmable deaminases. Nature Biotechnology, 35(5), 475-480. doi.org/10.1038/NBT.3852
Kim, H. J., Seo, S. W., Chang, J. W., Lee, J. Il, Kim, C. H., Chin, J., Choi, S. J., Kwon, H., Yun, H. J., Lee, J. M., Kim, S. T., Choe, Y. S., Lee, K. H., & Na, D. L. (2015). Stereotactic brain injection of human umbilical cord blood mesenchymal stem cells in patients with Alzheimer's disease dementia: A phase 1 clinical trial. Alzheimer's & Dementia (New York, N. Y.), 1(2), 95-102. doi.org/10.1016/J.TRCI.2015.06.007
Kim, S., Kim, D., Cho, S. W., Kim, J., & Kim, J. S. (2014). Highly efficient RNA-guided genome editing in human cells via delivery of purified Cas9 ribonucleoproteins. Genome Research, 24(6), 1012-1019. doi.org/10.1101/GR.171322.113
Kleinstiver, B. P., Prew, M. S., Tsai, S. Q., Topkar, V. V., Nguyen, N. T., Zheng, Z., Gonzales, A. P. W., Li, Z., Peterson, R. T., Yeh, J. R. J., Aryee, M. J., & Joung, J. K. (2015). Engineered CRISPR-Cas9 nucleases with altered PAM specificities. Nature, 523(7561), 481-485. doi.org/10.1038/NATURE14592
Koblan, L. W., Doman, J. L., Wilson, C., Levy, J. M., Tay, T., Newby, G. A., Maianti, J. P., Raguram, A., & Liu, D. R. (2018). Improving cytidine and adenine base editors by expression optimization and ancestral reconstruction. Nature Biotechnology, 36(9), 843-848. doi.org/10.1038/NBT.4172
Koblan, L. W., Erdos, M. R., Gordon, L. B., Collins, F. S., Brown, J. D., & Liu, D. R. (2021). Base editor treats progeria in mice. Nature. doi.org/10.1038/D41586-021-01114-8
Komor, A. C., Kim, Y. B., Packer, M. S., Zuris, J. A., & Liu, D. R. (2016). Programmable editing of a target base in genomic DNA without double-stranded DNA cleavage. Nature, 533(7603), 420-424. doi.org/10.1038/NATURE17946
Komor, A. C., Zhao, K. T., Packer, M. S., Gaudelli, N. M., Waterbury, A. L., Koblan, L. W., Kim, Y. B., Badran, A. H., & Liu, D. R. (2017a). Improved base excision repair inhibition and bacteriophage Mu Gam protein yields C:G-to-T:A base editors with higher efficiency and product purity. Science Advances, 3(8). doi.org/10.1126/SCIADV.AA04774
Komor, A. C., Zhao, K. T., Packer, M. S., Gaudelli, N. M., Waterbury, A. L., Koblan, L. W., Kim, Y. B., Badran, A. H., & Liu, D. R. (2017b). Improved base excision repair inhibition and bacteriophage Mu Gam protein yields C:G-to-T:A base editors with higher efficiency and product purity. Science Advances, 3(8). doi.org/10.1126/SCIADV.AA04774
Kondo, E., Horii, A., & Fukushige, S. (2001). The interacting domains of three MutL heterodimers in man: hILH1 interacts with 36 homologous amino acid residues within hILH3, hPMS1 and hPMS2. Nucleic Acids Research, 29(8), 1695-1702. doi.org/10.1093/NAR/29.8.1695
Kovalenko, M., Dragileva, E., St. Claire, J., Gillis, T., Guide, J. R., New, J., Dong, H., Kucherlapati, R., Kucherlapati, M. H., Ehrlich, M. E., Lee, J. M., & Wheeler, V. C. (2012). Msh2 acts in medium-spiny striatal neurons as an enhancer of CAG instability and mutant huntingtin phenotypes in Huntington's disease knock-in mice. PloS One, 7(9). doi.org/10.1371/JOURNAL.PONE.0044273
Kriegler, M. (1990). Gene Transfer and Expression. In Gene Transfer and Expression. doi.org/10.1007/978-1-349-11891-5
Larson, E., Fyfe, I., Morton, A. J., & Monckton, D. G. (2015). Age-, tissue- and length-dependent bidirectional somatic CAG⋅CTG repeat instability in an allelic series of R6/2 Huntington disease mice. Neurobiology of Disease, 76, 98-111. doi.org/10.1016/J.NBD.2015.01.004
Lee, J. M., Correia, K., Loupe, J., Kim, K. H., Barker, D., Hong, E. P., Chao, M. J., Long, J. D., Lucente, D., Vonsattel, J. P. G., Pinto, R. M., Abu Elneel, K., Ramos, E. M., Mysore, J. S., Gillis, T., Wheeler, V. C., MacDonald, M. E., Gusella, J. F., McAllister, B., . . . Myers, R. H. (2019). CAG Repeat Not Polyglutamine Length Determines Timing of Huntington's Disease Onset. Cell, 178(4), 887-900.e14. doi.org/10.1016/J.CELL.2019.06.036
Lee, J. M., Pinto, R. M., Gillis, T., St. Claire, J. C., & Wheeler, V. C. (2011). Quantification of age-dependent somatic CAG repeat instability in Hdh CAG knock-in mice reveals different expansion dynamics in striatum and liver. PloS One, 6(8). doi.org/10.1371/JOURNAL.PONE.0023647
Lee, J. M., Wheeler, V. C., Chao, M. J., Vonsattel, J. P. G., Pinto, R. M., Lucente, D., Abu-Elneel, K., Ramos, E. M., Mysore, J. S., Gillis, T., MacDonald, M. E., Gusella, J. F., Harold, D., Stone, T. C., Escott-Price, V., Han, J., Vedernikov, A., Holmans, P., Jones, L., . . . Myers, R. H. (2015). Identification of Genetic Factors that Modify Clinical Onset of Huntington's Disease. Cell, 162(3), 516-526. doi.org/10.1016/J.CELL.2015.07.003
Liang, X., Potter, J., Kumar, S., Zou, Y., Quintanilla, R., Sridharan, M., Carte, J., Chen, W., Roark, N., Ranganathan, S., Ravinder, N., & Chesnut, J. D. (2015). Rapid and highly efficient mammalian cell engineering via Cas9 protein transfection. Journal of Biotechnology, 208, 44-53. doi.org/10.1016/J.JBIOTEC.2015.04.024
Lim, C. K. W., Gapinske, M., Brooks, A. K., Woods, W. S., Powell, J. E., Zeballos C., M. A., Winter, J., Perez-Pinera, P., & Gaj, T. (2020). Treatment of a Mouse Model of ALS by In Vivo Base Editing. Molecular Therapy: The Journal of the American Society of Gene Therapy, 28(4), 1177-1189. doi.org/10.1016/J.YMTHE.2020.01.005
Lipkin, S. M., Moens, P. B., Wang, V., Lenzi, M., Shanmugarajah, D., Gilgeous, A., Thomas, J., Cheng, J., Touchman, J. W., Green, E. D., Schwartzberg, P., Collins, F. S., & Cohen, P. E. (2002). Meiotic arrest and aneuploidy in MLH3-deficient mice. Nature Genetics, 31(4), 385-390. doi.org/10.1038/NG931
Lokanga, R. A., Zhao, X. N., & Usdin, K. (2014). The mismatch repair protein MSH2 is rate limiting for repeat expansion in a fragile X premutation mouse model. Human Mutation, 35(1), 129-136. doi.org/10.1002/HUMU.22464
Loupe, J. M., Pinto, R. M., Kim, K. H., Gillis, T., Mysore, J. S., Andrew, M. A., Kovalenko, M., Murtha, R., Seong, I., Gusella, J. F., Kwak, S., Howland, D., Lee, R., Lee, J. M., Wheeler, V. C., & MacDonald, M. E. (2020). Promotion of somatic CAG repeat expansion by Fan1 knock-out in Huntington's disease knock-in mice is blocked by Mlh1 knock-out. Human Molecular Genetics, 29(18), 3044-3053. doi.org/10.1093/HMG/DDAA196
Miller, C. J., Kim, G. Y., Zhao, X., & Usdin, K. (2020). All three mammalian MutL complexes are required for repeat expansion in a mouse cell model of the Fragile X-related disorders. PLoS Genetics, 16(6). doi.org/10.1371/JOURNAL.PGEN.1008902
Miller, S. M., Wang, T., Randolph, P. B., Arbab, M., Shen, M. W., Huang, T. P., Matuszek, Z., Newby, G. A., Rees, H. A., & Liu, D. R. (2020). Continuous evolution of SpCas9 variants compatible with non-G PAMs. Nature Biotechnology, 38(4), 471-481. doi.org/10.1038/S41587-020-0412-8
Mingozzi, F., & High, K. A. (2011). Therapeutic in vivo gene transfer for genetic disease using AAV: progress and challenges. Nature Reviews. Genetics, 12(5), 341-355. doi.org/10.1038/NRG2988
Morrison, D. A. (1977). Transformation in Escherichia coli: cryogenic preservation of competent cells. Journal of Bacteriology, 132(1), 349-351. doi.org/10.1128/JB.132.1.349-351.1977
Moss, D. J. H., Tabrizi, S. J., Mead, S., Lo, K., Pardinas, A. F., Holmans, P., Jones, L., Langbehn, D., Coleman, A., Santos, R. D., Decolongon, J., Sturrock, A., Bardinet, E., Ret, C. J., Justo, D., Lehericy, S., Marelli, C., Nigaud, K., Valabregue, R., . . . Tan, L. (2017). Identification of genetic variants associated with Huntington's disease progression: a genome-wide association study. The Lancet. Neurology, 16(9), 701-711. doi.org/10.1016/S1474-4422(17)30161-8
Muzyczka, N. (1992). Use of adeno-associated virus as a general transduction vector for mammalian cells. Current Topics in Microbiology and Immunology, 158, 97-129. doi.org/10.1007/978-3-642-75608-5_5 Neering, S. J., Hardy, S. F., Minamoto, D., Spratt, S. K., & Jordan, C. T.
(1996). Transduction of Primitive Human Hematopoietic Cells With Recombinant Adenovirus Vectors. Blood, 88(4), 1147-1155. doi.org/10.1182/BLOOD.V88.4.1147.BLOODJOURNAL8841147
Nishant, K. T., Plys, A. J., & Alani, E. (2008). A mutation in the putative MLH3 endonuclease domain confers a defect in both mismatch repair and meiosis in Saccharomyces cerevisiae. Genetics, 179(2), 747-755. doi.org/10.1534/GENETICS.108.086645
Nishida, K., Arazoe, T., Yachie, N., Banno, S., Kakimoto, M., Tabata, M., Mochizuki, M., Miyabe, A., Araki, M., Hara, K. Y., Shimatani, Z., & Kondo, A. (2016). Targeted nucleotide editing using hybrid prokaryotic and vertebrate adaptive immune systems. Science (New York, N.Y.), 353(6305). doi.org/10. 1126/SCIENCE.AAF8729
Nishimasu, H., Ran, F. A., Hsu, P. D., Konermann, S., Shehata, S. I., Dohmae, N., Ishitani, R., Zhang, F., & Nureki, O. (2014). Crystal structure of Cas9 in complex with guide RNA and target DNA. Cell, 156(5), 935-949. doi.org/10.1016/J.CELL.2014.02.001
Oligino, T., Poliani, P. L., Wang, Y., Tsai, S. Y., O'Malley, B. W., Fink, D. J., & Glorioso, J. C. (1998). Drug inducible transgene expression in brain using a herpes simplex virus vector. Gene Therapy, 5(4), 491-496. doi.org/10.1038/SJ.GT.3300612
Owen, B. A. L., Yang, Z., Lai, M., Gajek, M., Badger, J. D., Hayes, J. J., Edelmann, W., Kucherlapati, R., Wilson, T. M., & McMurray, C. T. (2005). (CAG)(n)-hairpin DNA binds to Msh2-Msh3 and changes properties of mismatch recognition. Nature Structural & Molecular Biology, 12(8), 663-670. doi.org/10.1038/NSMB965
Palva, I., Lehtovaara, P., Kaariainen, L., Sibakov, M., Cantell, K., Schein, C. H., Kashiwagi, K., & Weissmann, C. (1983). Secretion of interferon by Bacillus subtilis. Gene, 22(2-3), 229-235. doi.org/10.1016/0378-1119(83)90107-5
Pinto, R. M., Arning, L., Giordano, J. V., Razghandi, P., Andrew, M. A., Gillis, T., Correia, K., Mysore, J. S., Grote Urtubey, D. M., Parwez, C. R., von Hein, S. M., Brent Clark, H., Nguyen, H. P., FOrster, E., Beller, A., Jayadaev, S., Dirk Keene, C., Bird, T. D., Lucente, D., . . . Wheeler, V. C. (2020). Patterns of CAG repeat instability in the central nervous system and periphery in Huntington's disease and in spinocerebellar ataxia type 1. Human Molecular Genetics, 29(15), 2551-2567. doi.org/10.1093/HMG/DDAA139
Pinto, R. M., Dragileva, E., Kirby, A., Lloret, A., Lopez, E., St. Claire, J., Panigrahi, G. B., Hou, C., Holloway, K., Gillis, T., Guide, J. R., Cohen, P. E., Li, G. M., Pearson, C. E., Daly, M. J., & Wheeler, V. C. (2013). Mismatch repair genes Mlh1 and Mlh3 modify CAG instability in Huntington's disease mice: genome-wide and candidate approaches. PLoS Genetics, 9(10). doi.org/10.1371/JOURNAL.PGEN.1003930
Porto, E. M., Komor, A. C., Slaymaker, I. M., & Yeo, G. W. (2020). Base editing: advances and therapeutic opportunities. Nature Reviews. Drug Discovery, 19(12), 839-859. doi.org/10.1038/S41573-020-0084-6
Ranjha, L., Anand, R., & Cejka, P. (2014). The Saccharomyces cerevisiae Mlh1-Mlh3 heterodimer is an endonuclease that preferentially binds to Holliday junctions. The Journal of Biological Chemistry, 289(9), 5674-5686. doi.org/10.1074/JBC.M113.533810
Rendahl, K. G., Leff, S. E., Otten, G. R., Spratt, S. K., Bohl, D., Van Roey, M., Donahue, B. A., Cohen, L. K., Mandel, R. J., Danos, O., & Snyder, R. O. (1998). Regulation of gene expression in vivo following transduction by two separate rAAV vectors. Nature Biotechnology, 16(8), 757-761. doi.org/10.1038/NBT0898-757
Rogacheva, M. V., Manhart, C. M., Chen, G., Guarne, A., Surtees, J., & Alani, E. (2014). Mlh1-Mlh3, a meiotic crossover and DNA mismatch repair factor, is a Msh2-Msh3-stimulated endonuclease. The Journal of Biological Chemistry, 289(9), 5664-5673. doi.org/10.1074/JBC.M113.534644
Roy, J. C. L., Vitalo, A., Andrew, M. A., Mota-Silva, E., Kovalenko, M., Burch, Z., Nhu, A. M., Cohen, P. E., Grabczyk, E., Wheeler, V. C., & Mouro Pinto, R. (2021). Somatic CAG expansion in Huntington's disease is dependent on the MLH3 endonuclease domain, which can be excluded via splice redirection. Nucleic Acids Research, 49(7), 3907-3918. doi.org/10.1093/NAR/GKAB152
Sambrook, J., & Russell, D. W. (2001). Molecular Cloning: A Laboratory Manual. Third Edition. In United States: Cold Spring Harbor Laboratory Press.
Savouret, C., Brisson, E., Essers, J., Kanaar, R., Pastink, A., Te Riele, H., Junien, C., & Gourdon, G. (2003). CTG repeat instability and size variation timing in DNA repair-deficient mice. The EMBO Journal, 22(9), 2264-2273. doi.org/10.1093/EMBOJ/CDG202
Schweitzer, J. S., Song, B., Herrington, T. M., Park, T.-Y., Lee, N., Ko, S., Jeon, J., Cha, Y., Kim, K., Li, Q., Henchcliffe, C., Kaplitt, M., Neff, C., Rapalino, O., Seo, H., Lee, I.-H., Kim, J., Kim, T., Petsko, G. A., . . . Kim, K.-S. (2020). Personalized iPSC-Derived Dopamine Progenitor Cells for Parkinson's Disease. The New England Journal of Medicine, 382(20), 1926-1932. doi.org/10.1056/NEJMOA1915872
She, K., Liu, Y., Zhao, Q., Jin, X., Yang, Y., Su, J., Li, R., Song, L., Xiao, J., Yao, S., Lu, F., Wei, Y., & Yang, Y. (2023). Dual-AAV split prime editor corrects the mutation and phenotype in mice with inherited retinal degeneration. Signal Transduction and Targeted Therapy, 8(1). doi.org/10.1038/S41392-022-01234-1
Shelbourne, P. F., Keller-McGandy, C., Bi, W. L., Yoon, S. R., Dubeau, L., Veitch, N. J., Vonsattel, J. P., Wexler, N. S., Norman, A., & Augood, S. J. (2007). Triplet repeat mutation length gains correlate with cell-type specific vulnerability in Huntington disease brain. Human Molecular Genetics, 16(10), 1133-1142. doi.org/10.1093/HMG/DDM054
Swami, M., Hendricks, A. E., Gillis, T., Massood, T., Mysore, J., Myers, R. H., & Wheeler, V. C. (2009). Somatic expansion of the Huntington's disease CAG repeat in the brain is associated with an earlier age of disease onset. Human Molecular Genetics, 18(16), 3039-3047. doi.org/10.1093/HMG/DDP242
Thuronyi, B. W., Koblan, L. W., Levy, J. M., Yeh, W. H., Zheng, C., Newby, G. A., Wilson, C., Bhaumik, M., Shubina-Oleinik, O., Holt, J. R., & Liu, D. R. (2019). Continuous evolution of base editors with expanded target compatibility and improved activity. Nature Biotechnology, 37(9), 1070-1079. doi.org/10.1038/S41587-019-0193-0
Toledo, M., Sun, X., Brieno-Enriquez, M. A., Raghavan, V., Gray, S., Pea, J., Milano, C. R., Venkatesh, A., Patel, L., Borst, P. L., Alani, E., & Cohen, P. E. (2019). A mutation in the endonuclease domain of mouse MLH3 reveals novel roles for MutLγ during crossover formation in meiotic prophase I. PLoS Genetics, 15(6). doi.org/10.1371/JOURNAL.PGEN.1008177
Van Den Broek, W. J. A. A., Nelen, M. R., Wansink, D. G., Coerwinkel, M. M., Te Riele, H., Groenen, P. J. T. A., & Wieringa, B. (2002). Somatic expansion behaviour of the (CTG)n repeat in myotonic dystrophy knock-in mice is differentially affected by Msh3 and Msh6 mismatch-repair proteins. Human Molecular Genetics, 11(2), 191-198. doi.org/10.1093/HMG/11.2.191
Villiger, L., Grisch-Chan, H. M., Lindsay, H., Ringnalda, F., Pogliano, C. B., Allegri, G., Fingerhut, R., Haberle, J., Matos, J., Robinson, M. D., Thony, B., & Schwank, G. (2018). Treatment of a metabolic liver disease by in vivo genome base editing in adult mice. Nature Medicine, 24(10), 1519-1525. doi.org/10.1038/S41591-018-0209-1
Vonsattel, J. P., Myers, R. H., Stevens, T. J., Ferrante, R. J., Bird, E. D., & Richardson, E. P. (1985). Neuropathological classification of Huntington's disease. Journal of Neuropathology and Experimental Neurology, 44(6), 559-577. doi.org/10.1097/00005072-198511000-00003
Walton, R. T., Christie, K. A., Whittaker, M. N., & Kleinstiver, B. P. (2020a). Unconstrained genome targeting with near-PAMless engineered CRISPR-Cas9 variants. Science (New York, N.Y.), 368(6488), 290-296. doi.org/10.1126/SCIENCE.ABA8853
Walton, R. T., Christie, K. A., Whittaker, M. N., & Kleinstiver, B. P. (2020b). Unconstrained genome targeting with near-PAMless engineered CRISPR-Cas9 variants. Science (New York, N.Y.), 368(6488), 290-296. doi.org/10.1126/SCIENCE.ABA8853
Wang, Y., Xu, J., Pierson, T., O'Malley, B. W., & Tsai, S. Y. (1997). Positive and negative regulation of gene expression in eukaryotic cells with an inducible transcriptional regulator. Gene Therapy, 4(5), 432-441. doi.org/10.1038/SJ.GT.3300402
Wheeler, V. C., Lebel, L. A., Vrbanac, V., Teed, A., te Riele, H. T., & MacDonald, M. E. (2003). Mismatch repair gene Msh2 modifies the timing of early disease in Hdh(Q111) striatum. HumanMolecular Genetics, 12(3), 273-281. doi.org/10.1093/HMG/DDG056
Wright, G. E. B., Collins, J. A., Kay, C., McDonald, C., Dolzhenko, E., Xia, Q., Beĉanović, K., Drogemoller, B. I., Semaka, A., Nguyen, C. M., Trost, B., Richards, F., Bijlsma, E. K., Squitieri, F., Ross, C. J. D., Scherer, S. W., Eberle, M. A., Yuen, R. K. C., & Hayden, M. R. (2019). Length of Uninterrupted CAG, Independent of Polyglutamine Size, Results in Increased Somatic Instability, Hastening Onset of Huntington Disease. American Journal of Human Genetics, 104(6), 1116-1126. doi.org/10.1016/J.AJHG.2019.04.007
Yates, A. D., Achuthan, P., Akanni, W., Allen, J., Allen, J., Alvarez-Jarreta, J., Amode, M. R., Armean, I. M., Azov, A. G., Bennett, R., Bhai, J., Billis, K., Boddu, S., Marugin, J. C., Cummins, C., Davidson, C., Dodiya, K., Fatima, R., Gall, A., . . . Flicek, P. (2020). Ensembl 2020. Nucleic Acids Research, 48(D1), D682-D688. doi.org/10.1093/NAR/GKZ966
Zhao, X. N., Lokanga, R., Allette, K., Gazy, I., Wu, D., & Usdin, K. (2016). A MutSβ-Dependent Contribution of MutSα to Repeat Expansions in Fragile X Premutation Mice? PLoS Genetics, 12(7). doi.org/10.1371/JOURNAL.PGEN.1006190
Zhao, X., Zhang, Y., Wilkins, K., Edelmann, W., & Usdin, K. (2018). MutLγ promotes repeat expansion in a Fragile X mouse model while EXO1 is protective. PLoS Genetics, 14(10). doi.org/10.1371/JOURNAL.PGEN.1007719
Zuris, J. A., Thompson, D. B., Shu, Y., Guilinger, J. P., Bessen, J. L., Hu, J. H., Maeder, M. L., Joung, J. K., Chen, Z. Y., & Liu, D. R. (2015). Cationic lipid-mediated delivery of proteins enables efficient protein-based genome editing in vitro and in vivo. Nature Biotechnology, 33(1), 73-80. doi.org/10.1038/NBT.3081

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A method of reducing expansion of nucleotide repeats, optionally an expansion of CAG trinucleotide repeats in a huntingtin (HTT) gene, in a cell, the method comprising contacting the cell with or expressing in the cell a base editor enzyme and a guide RNA (gRNA) that directs the base editor to a splice acceptor site for mutL homolog 3 (MLH3) exon 7 or MLH3 endonuclease domain in an amount sufficient to reduce the expansion of nucleotide repeats in the cell.

2. A method of treating a subject who has a condition associated with nucleotide repeat expansion, optionally CAG nucleotide repeat expansion in a HTT gene, the method comprising administering to the subject a therapeutically effective amount of a base editor and a guide RNA that directs the base editor to MLH3, preferably wherein the gRNA binds to an exon/repeat border or endonuclease domain, in an amount sufficient to reduce the expansion of nucleotide repeats in the cell.

3. The method of claim 2, wherein the base editor and gRNA are administered to the CNS, optionally to the brain or spinal cord of the subject (optionally via ICV, cisternae magna, or intrathecal administration), or administered systemically to the subject.

4. The method of claim 1, wherein the base editor comprises Cas9, optionally wild type SpCas9, SpG, SpRY, VRQR, SpNG, SpNRRH, SpNRCH, or SpNRTH.

5. The method of claim 4, wherein the base editor comprises a nicking or catalytically inactive wild type SpCas9, SpG, SpRY, VRQR, SpNG, SpNRRH, SpNRCH, or SpNRTH and a UGI or TadA.

6. The method of claim 1, wherein the gRNA comprises a spacer sequence listed in Table 1, optionally truncated to 19, 18, or 17 nt by removing 1, 2, or 3 bases from the 3′ PAM distal end of the spacer.

7. The method of claim 6, wherein the gRNA comprises a spacer sequence SA-A4, SA-A5, or SA-A6, optionally truncated to 19, 18, or 17 nt by removing 1, 2, or 3 bases from the 3′ PAM distal end of the spacer.

8. The method of claim 1, wherein the base editor and gRNA are administered in an expression vector, optionally a plasmid or viral vector; are administered as mRNA; or are administered as RNPs.

9. A composition comprising a base editor and gRNA, wherein the gRNA comprises a spacer sequence having 17-20 nts of SA-A4, SA-A5, or SA-A6, optionally wherein the base editor comprises Cas9, optionally wild type SpCas9, SpG, SpRY, VRQR, SpNG, SpNRRH, SpNRCH, or SpNRTH or nicking or catalytically inactive wild type SpCas9, SpG, SpRY, VRQR, SpNG, SpNRRH, SpNRCH, or SpNRTH and a UGI or TadA.

10. The composition of claim 9, wherein the base editor and gRNA are:
ABE8e-SpG and SA-A6;
ABE8e-SpG and SA-A4;
ABE8e-SpRY and SA-A5;
ABE8e-SpNRCH and SA-A4;
ABE8e-SpNRCH and SA-A6;
ABE8e-SpNG and SA-A5;
ABE8e-SpNG and SA-A6;
ABE8e-SpNRTH and SA-A4; or
ABE8e-SpNRTH and SA-A6.

11. The composition of claim 9, wherein the base editor and gRNA are in ribonucleoprotein complexes.

12. A nucleic acid encoding a base editor and gRNA, wherein the gRNA comprises a spacer sequence having 17-20 nts of SA-A4, SA-A5, or SA-A6.

13. The nucleic acid of claim 12, wherein the base editor comprises Cas9, optionally wild type SpCas9, SpG, SpRY, VRQR, SpNG, SpNRRH, SpNRCH, or SpNRTH or nicking or catalytically inactive wild type SpCas9, SpG, SpRY, VRQR, SpNG, SpNRRH, SpNRCH, or SpNRTH and a UGI or TadA.

14. The nucleic acid of claim 12, wherein the base editor and gRNA are:
ABE8e-SpG and SA-A6;
ABE8e-SpG and SA-A4;
ABE8e-SpRY and SA-A5;
ABE8e-SpNRCH and SA-A4;
ABE8e-SpNRCH and SA-A6;
ABE8e-SpNG and SA-A5;
ABE8e-SpNG and SA-A6;
ABE8e-SpNRTH and SA-A4; or
ABE8e-SpNRTH and SA-A6.

15. The nucleic acid of claim 12, wherein the nucleic acid is in an expression vector.

16. The nucleic acid of claim 14, wherein the expression vector is a plasmid or viral vector, and further comprises at least one promoter that drives expression of the base editor and gRNA.

17. The nucleic acid of claim 12, comprising mRNA encoding the base editor and gRNA.
